(19) United States
(12) Patent Application Publication
(10) Pub. No.: US 2024/0358837 A1
(43) Pub. Date: Oct. 31, 2024

(54) Amorphous Photosensitizing Particles, Methods For The Preparation Thereof, And Methods For The Use Thereof

(71) Applicant: University of Maryland, College Park, College Park, MD (US)

(72) Inventors: Huang-Chiao Huang, College Park, MD (US); Collin Inglut, College Park, MD (US)

(21) Appl. No.: 18/692,813

(22) PCT Filed: Sep. 16, 2022
(86) PCT No.: PCT/US2022/076586

§ 371 Date: Mar. 16, 2024

Related U.S. Application Data
(60) Provisional application No. 63/245,733, filed on Sep. 17, 2021.

(51) Int. Cl. A61K 41/00; A61K 9/16; A61P 35/00

(52) U.S. Cl. CPC A61K 41/0071; A61K 9/16; A61K 9/1688; A61P 35/00

(57) ABSTRACT

Amorphous nanoparticle compositions comprising a photosensitizer along with methods of making and using the same are provided. In particular, the amorphous nanoparticle compositions are generated and used in carrier-free, solubilizing agent-free applications to improve the photophysical and photochemical stability of clinically used photosensitizers, particularly verteporfin. The amorphous nanoparticle compositions may be used in fluorescence-guided surgery, photodynamic therapy of cancer and non-cancer diseases, fluorescence diagnosis of cancer and non-cancer diseases, blood-brain barrier opening, drug delivery to the brain, and/or in various other types of treatments and implementations.

### CROSS-REFERENCE TO RELATED APPLICATIONS

This application claims priority and is related to U.S. Provisional Application Ser. No. 63/245,733, filed on Sep. 17, 2021. The entire contents of this application are hereby expressly incorporated herein by reference including, without limitation, the specification, claims, and abstract, as well as any figures, tables, and drawings thereof.

### TECHNICAL FIELD

The present disclosure generally relates to photosensitizers and the use of photosensitizers, for example, in photochemistry-based treatments and fluorescence imaging. Examples of the present disclosure provide novel amorphous, photosensitizing particles and methods of making such particles and utilizing such particles, for example, in the treatment and imaging of disease. In some examples, such particles may be used in association with a carrier-free solubilizing agent-free implementation to improve the photophysical and photochemical stability of clinically used photosensitizers (e.g., verteporfin, also known as BPD). In some examples, such particles may be used in fluorescence-guided surgery, photodynamic therapy of cancer and non-cancer diseases, fluorescence diagnosis of cancer and non-cancer diseases, blood-brain barrier opening, drug delivery to the brain, and/or in various other types of treatments and implementations.

### TECHNICAL BACKGROUND

Photodynamic therapy (PDT) involves the light activation of non-toxic photosensitizers (PS) to modulate tissues or induce cell death through the production of reactive oxygen species (ROS). This photochemistry-based treatment modality has shown promise in treating different ailments, ranging from wet age-related macular degeneration to refractory brain tumors and pancreatic ductal adenocarcinoma. To enhance prognosis, PDT is typically combined with conventional treatments, such as surgery, chemotherapy, immunotherapy, and radio chemotherapy. PDT has the ability to inflict direct damage to subcellular organelles (e.g., mitochondria, endoplasmic reticulum, lysosome), bypass cell-death signaling pathways required for chemotherapy, and does not result in overlapping side effects. PDT is also capable of permeabilizing tumor vasculature and the BBB, enhancing intratumoral drug delivery.

Photosensitizers are drugs that respond to optical stimuli and generate chemical reactions with spatiotemporal precision to induce targeted biological changes. A variety of photosensitizers have been investigated and marketed for PDT of actinic keratosis, choroidal neovascularization, bacterial infections, and cancer. Since the U.S. Food and Drug Administration (FDA) approved the first nano-formulation of photosensitizer in 2000—a Liposomal verteporfin—for PDT of macular degeneration, significant advancements have been made in the development of photosensitizer delivery. Verteporfin (VP, also known as benzoporphyrin derivative) is a photosensitizer with excellent singlet oxygen yield for photodynamic therapy (PDT) and minimal systemic toxicities due to rapid clearance primarily via liver-mediated mechanisms. PDT involves the intravenous administration of VP followed by non-thermal 690 nm laser activation of VP to generate highly cytotoxic, short-lived ROS. While VP has proven advantageous in a wide range of disease contexts, such as chorioretinal conditions and cancers, the molecule is relatively hydrophobic and has required carrier systems for clinical delivery.

Further, while liposomes significantly increase the ability of water-insoluble photosensitizers to achieve a clinically relevant concentration in aqueous media, they are necessarily enclosed by phospholipid bilayers that can potentially hinder the uptake and photochemical activity of photosensitizers in cells. This encapsulation in a lipid bilayer results in a limited loading capacity and an increased risk of photosensitizer self-quenching—a bottleneck for an effective PDT in the clinic. Thus, although liposomes have considerable clinical value, they still have important limitations when used for photosensitizer delivery. Accordingly, there is a need to improve the cell-level delivery of photosensitizers. Relatedly, there is a need to develop a carrier-free nanoformulation with efficient photosensitizer delivery and release for enhanced PDT.

Besides their use in PDT treatment, PS is used in a variety of imaging applications. The activated PS emits a fluorescence signal which can be used in fluorescence-guided resection (FGR) of tumors. One particularly successful PS for the FGR of glioblastoma is 5-ALA-induced protoporphyrin IX (PpIX). After oral administration of the prodrug 5-ALA (Gleolan®), gliomas that contain PpIX emit a violet-red fluorescence upon excitation with ultraviolet/blue light (375-440 nm), helping guide surgeons during tumor resection. While the use of this FDA-approved surgical technique has shown to increase the mean patient survival by 3 months compared to normal white light surgery, FGR and ALA-mediated PDT have not yet achieved long-term survival. There is therefore a need to improve the delivery mechanisms of photosensitizers.

There have been many attempts to improve the liposomal delivery of chemotherapeutics and PS. In some cases, covalent conjugation of PS to biomolecules (e.g., lipids, polymers) can enhance stability post-injection, allow for the targeting of different organelles, escape drug efflux, and improve imaging.

Similarly, the FDA-approved anticancer liposome formulations. Doxil and Onivyde, have been successful due in part to, their unique solidified drug core (i.e., crystal and gel phase), that can minimize cardiotoxicities and increase maximum tolerable doses. Yet, the significant drawbacks of liposomes have not been overcome: encapsulation of therapeutics within liposomes has been shown to alter the drug biodistribution via blood serum protein adsorption. Additionally, liposomes tend to largely accumulate within the liver and spleen (i.e., organs that contain the mononuclear phagocyte system, MPS), where they can result in macrophage depletion and phagocytic capacity impairment. In addition, liposome formulations containing polyethylene glycol (PEG) can also stimulate the formation and release of anti-PEG IgM antibodies from B cells within the spleen. Repeated doses of pegylated liposomes can lose their stealth ability and are more quickly removed from blood circulation. These limitations highlight the need for a hydrophobic PS delivery platform that can be intravenously administered without a solubilizing vehicle or a liposome carrier.

Nanocrystal drug formulations have been proposed as a potential solution to overcome the challenges associated with water-insoluble therapeutics and liposomal delivery. Nanocrystals are typically used for the oral administration of hydrophobic drugs with low bioactivity, for example, Gris-PEG® (griscofulvin), Naprelan® (naproxen sodium), and Zelboraf® (Vemurafenib). Carrier-free nanodispersions can also be administered intravenously. Overall, nanocrystal formulations allow for a theoretical drug loading of 100%, eliminating the reliance on lipids and cosolvents, while also being easily synthesized via media milling or reprecipitation.

Nanoconstructs having a crystalline structure have received more focus because they are very stable, have a high saturation solubility, and have high stability post intravenous injection due to the minimized Gibbs free energy. Such nanocrystals have been leveraged for extended-release of antiretroviral therapies, anticancer drugs, and other medications. However, particularly in the context of PDT, the potential for prolonged photosensitizer bioavailability from nanocrystals could be unfavorable in terms of skin phototoxicity.

In comparison, as described herein, amorphous nano drugs generally possess a higher saturation solubility and. consequently. an increased dissolution velocity (creation of high Cₘₐₓ, reduction of tₘₐₓ) compared to equally sized nanocrystals, thereby allowing for quicker drug release. The reduction back into single molecules during circulation can also help minimize the sequestration of nanocrystals by the MPS. The effective use of amorphous structures as described herein is surprising at least in part because amorphous nanodispersions are usually not kinetically stable and typically require an additional stabilizing agent (e.g., a polymer stabilizer).

There is therefore a need to develop stable amorphous nanoconstructs and methods of making the same. In particular, there is a need to engineer and characterize pure-drug VP amorphous nanoparticles that demonstrate equivalent or superior performance compared to a clinically relevant liposome formulation of VP (L-VP) and free form VP. There is a still further need to develop amorphous nanoconstructs and methods of making the same without the use of a stabilizing agent.

There also remains a broader need to develop carrier-free nano drugs capable of delivering higher levels of hydrophobic photosensitizers and which allow a theoretical drug loading capacity of 100%, eliminating the reliance on lipids, polymers, and co-solvents for drug delivery.

A further need exists to develop carrier-free, amorphous photosensitizer products for PDT with a stability that can be maintained throughout the product's shelf-life while maintaining their capability to be light-activated for ROS generation.

These and other objects, advantages, and features of the present disclosure will become apparent from the following specification taken in conjunction with the claims set forth herein.

### BRIEF SUMMARY

The present disclosure relates to nanoparticle compositions comprising a plurality of amorphous nanoparticles comprising a photosensitizer. Relatedly provided are nanoparticle compositions generated by a process comprising: (1) preparing a first liquid phase solution comprising a photosensitizer in a solvent; (2) preparing a second liquid phase solution comprising an antisolvent; (3) adding the first liquid phase solution dropwise into the second liquid phase solution; and (4) removing any remaining solvent, antisolvent, and a photosensitizer to produce amorphous nanoparticles. In an embodiment, step (3) further comprises the addition of energy through mixing, sonication, homogenization, countercurrent flow homogenization, microfluidization, or a combination thereof.

In an embodiment, the photosensitizer comprises 100 wt. % of the amorphous nanoparticles. In a further embodiment, the photosensitizer is present in the first liquid phase solution in a concentration of between about 100 micromolar (μM) to about 100 millimolar (mM).

According to some embodiments, the ratio between the solvent and the antisolvent is between about 1:50 to about 1:4.

In some embodiments, the antisolvent comprises distilled water, or other aqueous solutions. In further embodiments, the solvent comprises dimethylsulfoxide, (DMSO), N-methyl-2-pyrrolidinone (NMP), 2-pyrrolidinone, 1,3-dimethylimidazolidinone (DMI), dimethylacetamide (DMA), dimethylformamide (DMF), dioxane, acetone, tetrahydrofuran (THF), tetramethylenesulfone (sulfolane), acetonitrile, hexamethylphosphoramide (HMPA), nitromethane, ethanol, methanol, or a combination thereof.

In an embodiment, the photosensitizer is soluble in the solvent. According to an embodiment, the photosensitizer can absorb light in a range of about 400 nm to about 1200 nm. In a further embodiment, the photosensitizer comprises a hacmotoporphyrin, photofrin, porfimer sodium, chlorin, chlorin e6, bacteriochlorin, phthalocyanine, benzoporphyrin, purpurin. porphycene, pheophorbide, pheophorbide a, pyropheophorbide a methyl ester (MPPa), protoporphyrin IX (PpIX), meso-tetra (3-hydroxyphenyl) porphyrin (m-THPP), meso-tetra (3-hydroxyphenyl)chlorin (m-THPC), verdin, psoralen, or a combination thereof. In a preferred embodiment, the photosensitizer comprises verteporfin or a derivative thereof. In some embodiments, the compositions further comprise an excipient, disintegrant, lubricant, penetration enhancer, pH modifier, pH buffer, surfactant, or a combination thereof.

Disclosed herein are nanoparticle compositions comprising a plurality of amorphous nanoparticles comprising up to 100% by weight of a photosensitizer; wherein the amorphous nanoparticles have a size of 1000 nm or less; and wherein the amorphous nanoparticles are carrier-free and lipid-free. In a preferred embodiment, the photosensitizer comprises 100 wt. % of the amorphous nanoparticles.

According to an embodiment, the photosensitizer is soluble in the solvent. In a further embodiment, the photosensitizer absorbs light in a range of from about 400 nm to about 1200 nm. In a still further embodiment, the photosensitizer comprises a hacmotoporphyrin, photofrin, porfimer sodium, chlorin, chlorine e6, bacteriochlorin, phthalocyanine, benzoporphyrin, purpurin, porphycene, pheophorbide, pheophorbide, pheophorbide a, pyropheophorbide a methyl ester (MPPa), protoporphyrin IX (PpIX), meso-tetra (3-hydroxyphenyl) porphyrin (m-THPP), meso-tetra(3-hydroxyphenyl)chlorin (m-THPC), verdin, psoralen, or a combination thereof. In a preferred embodiment, the photosensitizer comprises verteporfin or a derivative thereof.

Additionally disclosed herein are methods of treating a disease comprising using photodynamic therapy comprising: (1) administering to a target location a composition comprising a plurality of amorphous nanoparticles comprising a photosensitizer in an amount effective to facilitate photodynamic therapy; and (2) exposing the amorphous nanoparticles to photoactivating light having a wavelength capable of being absorbed by the photosensitizer; thereby (3) producing cytotoxic reactive oxygen species at the target location.

Further provided are methods of treating a disease comprising: administering to a target an effective amount of a composition comprising amorphous nanoparticles comprising a photosensitizer.

In an embodiment, the target location is a tumor, a tissue, a cell, a vessel, or a combination thereof. In a further embodiment, the tumor is a glioblastoma. According to some embodiments, the disease is peritoneal cancer, liver cancer, pancreatic cancer, brain cancer, neck cancer, spinal cancer, lung cancer, prostate cancer, bladder cancer, skin cancer, eye cancer, oral cancer, head and neck cancer, blood cancer, bone cancer, stomach cancer, kidney cancer, breast cancer, colorectal cancer, cervical cancer, ovarian cancer, central nervous system tumor, or a combination thereof.

In an embodiment, the methods further comprise a step of administering an antineoplastic drug or a secondary therapeutic. In some embodiments, the compositions further comprise an antineoplastic drug, secondary therapeutic, excipient, disintegrant, lubricant, penetration enhancer, pH modifier, pH buffer, surfactant, or a combination thereof.

According to an embodiment, the administration is oral, mucosal, parenteral, or transdermal.

In an embodiment, the antineoplastic drug comprises cmaxanib, cyclosporin, etanercept, doxycycline, bortezomib, acivicin, aclarubicin, acodazole hydrochloride, acronine, adozelesin, aldesleukin, altretamine, ambomycin, ametantrone acetate, amsacrine, anastrozole, anthramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium. bropirimine, busulfan, cactinomycin, calusterone, caracemide, carbetimer, carboplatin, carmustine, carubicin hydrochloride, carzelesin, cedefingol, celecoxib, chlorambucil, cirolemycin, cisplatin, cladribine, crisnatol mesylate, cyclophosphamide, cytarabine, dacarbazine, dactinomycin, daunorubicin hydrochloride, decitabine, dexormaplatin, dezaguanine, dezaguanine mesylate, diaziquone, docetaxel, doxorubicin, doxorubicin hydrochloride, droloxifene, droloxifene citrate, dromostanolone propionate, duazomycin, edatrexate, eflomithine hydrochloride, elsamitrucin, enloplatin, enpromate, epipropidine, epirubicin hydrochloride, erbulozole, esorubicin hydrochloride, estramustine, estramustine phosphate sodium, ctanidazole, etoposide, ctoposide phosphate, etoprine, fadrozole hydrochloride, fazarabine, fenretinide, floxuridine, fludarabine phosphate, fluorouracil, flurocitabine, fosquidone, fostriecin sodium, gemcitabine, gemcitabine hydrochloride, hydroxyurea, idarubicin hydrochloride, ifosfamide, ilmofosine, iproplatin, irinotecan, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitogillin, mitomalcin, mitomycin, mitosper, mitotane, mitoxantrone hydrochloride, mycophenolic acid, nocodazole, nogalamycin, ormaplatin, oxisuran, paclitaxel, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, riboprine, safingol, safingol hydrochloride, semustine, simtrazene, sparfosate sodium, sparsomycin, spirogermanium hydrochloride, spiromustine, spiroplatin, streptonigrin, streptozocin, sulofenur, talisomycin, tecogalan sodium, taxotere, tegafur, teloxantrone hydrochloride, temoporfin, teniposide, teroxirone, testolactone, thiamiprine, thioguanine, thiotepa, tiazofurin, tirapazamine, toremifene citrate, trestolone acetate, triciribine phosphate, trimetrexate, trimetrexate glucuronate, triptorelin, tubulozole hydrochloride, uracil mustard, uredepa, vapreotide, vinblastine sulfate, vincristine sulfate, vindesine, vindesine sulfate, vinepidine sulfate, vinglycinate sulfate, vinleurosine sulfate, vinorelbine tartrate, vinrosidine sulfate, vinzolidine sulfate, vorozole, zeniplatin, zinostatin, zorubicin hydrochloride, or a combination thereof.

While multiple embodiments are disclosed, still other embodiments of the present disclosure will become apparent based on the detailed description, which shows and describes illustrative embodiments of the disclosure. The foregoing summary is illustrative only and is not intended to be in any way limiting. In addition to the illustrative aspects, embodiments, and features described above, further aspects, embodiments, and features of the present technology are apparent from the following drawings and the detailed description, which shows and describes illustrative embodiments of the present technology. Each feature of the technology described herein may be combined with any one or more other features of the disclosure, e.g., the methods may be used with any composition described herein. Accordingly, the drawings and detailed description are to be regarded as illustrative and not restrictive.

### BRIEF DESCRIPTION OF THE DRAWINGS

FIG. 1A shows a representative digital image of free-form VP, Liposomal VP, and NanoVP in phosphate-buffered saline (PBS).

FIG. 1B is an example schematic depiction of the solvent-antisolvent precipitation method for the preparation of NanoVP.

FIG. 1C depicts representative transmission electron microscopy (TEM) micrographs (Scale bar=200 nm).

FIG. 1D is a close-up view of monodispersed NanoVP.

FIG. 1F shows a representative image of a selected area electron diffraction of the amorphous NanoVP.

FIG. 1G depicts representative intensity plots, the average hydrodynamic diameter, and the average zeta potential of NanoVP diameter pre and post-dialysis. The hydrodynamic diameter and polydispersity index (PdI) of NanoVP.

FIG. 1H shows polydispersity index (PdI) and hydrodynamic diameter (nm) as a function of initial VP concentration in DMSO.

FIG. 1I shows polydispersity index (PdI) and hydrodynamic diameter (nm) as a function of DMSO:Water ratio. N≥3. Error bars show the standard error of the mean.

FIG. 2 shows a size quantification of NanoVP through TEM micrograph images. The black line in the graph shows the Gaussian distribution curve of the data set. Bin size=10 nm, N=250 particles.

FIG. 3 shows an image of a representative ionic liquid TEM micrograph. A second electron imaging technique was used to verify that the “drying effect” did not impact NanoVP structure or morphology. Scale bar=1000 nm.

FIG. 4 shows the results of alternative NanoVP synthesis methods. In particular, considering the test tubes and viewing left to right, the image depicts (1) VP lyophilized powder added to PBS, which did dissolve. Fully dissolved VP in DMSoOwas also added into PBS (2) dropwise and (3) non-dropwise. Adding VP solution directly into PBS results in uncontrolled VP aggregation. The image also shows (4) NanoVP synthesis following established precipitation in water, along with (5) NanoVP post-dialysis in PBS.

FIG. 5A is an intensity plot of NanoVP diameter (nm) as a function of the DMSO: water ratio.

FIG. 5B is an intensity plot of NanoVP diameter (nm) as a function of the initial VP concentration.

FIG. 6A is a graph showing NanoVP diameter and PdI in PBS.

FIG. 6B is an intensity plot showing NanoVP intensity in water.

FIG. 7 shows the results of evaluating NanoVP electrostatic stabilization, in particular representative digital images of NanoVP coated polymers. NanoVP was synthesized with (left) neutral polymer poloxamer 407 or (right) cationic polymer polyethyleneimine. Poloxamer 407 had no impact on NanoVP stability, size, or PdI. Polyethylenimine caused NanoVP to form large aggregates.

FIG. 8A describes the photochemical characterization of NanoVP, free-form VP, and liposomal VP, in particular a representative absorbance and fluorescence (FL) spectra of 5 μM NanoVP, free-form VP, and liposomal VP in DMSO. VP was excited at excitation 435±10 nm.

FIG. 8B describes the photochemical characterization of NanoVP, free-form VP, and liposomal VP, in particular representative absorbance spectra of NanoVP, free-form VP, and liposomal VP in PBS (red) and DMSO (blue).

FIG. 8C describes the photochemical characterization of NanoVP, free-form VP, and liposomal VP, in particular fluorescence quenching, the maximum fluorescence intensity of 0.625-5 μM NanoVP, free-form VP, and liposomal VP in DMSO (FLDMSO) divided by the maximum intensity within PBS (FL₀).

FIG. 8D describes the photochemical characterization of NanoVP, free-form VP, and liposomal VP, in particular singlet oxygen production from NanoVP, free-form VP, and liposomal VP-mediated PDT (10 J/cm², 10 mW/cm²).

FIG. 8E describes the photochemical characterization of NanoVP, free-form VP, and liposomal VP, in particular, fluorescence quenching of NanoVP as a function of serum concentration in PBS.

FIG. 8F describes the photochemical characterization of NanoVP, free-form VP, and liposomal VP, in particular the intracellular ROS production from NanoVP, free-form VP, and Liposomal VP-mediated PDT (10 J/cm², 50 mW/cm²). One-way ANOVA with multiple comparison test was used to calculate significant differences, where * P<0.05, ** P<0.01, *** P<0.001. N≥3. Error bar shows the standard error of the mean.

FIG. 9 shows high power light activation of VP results in limited photothermal effects, wherein NanoVP, freeform VP, or liposomal VP (40 μM) in PBS were light-activated (50 J/cm², 0.5 W/cm²) and solution temperature was measured. Error bar shows the standard error of the mean.

FIG. 10A shows NanoVP fluorescence recovers in DMSO, PBS, and cell culture medium, in particular the time-dependent fluorescence signal (Excitation/Emission: 435/700) of 5 μM in PBS, DMSO, and complete cell culture medium supplemented with 10% FBS at 37° C.

FIG. 10B shows the time-dependent normalized fluorescence signal of NanoVP, free-form VP, and liposomal VP when incubated with a monolayer of U87 cells within a cell culture medium over a 3-hour period at 37° C.

FIG. 10C shows the time-dependent normalized fluorescence signal of NanoVP, free-form VP, and liposomal VP when incubated with a monolayer of U87 cells within PBS over a 3-hour period at 37° C.

FIG. 11A shows the in vitro PDT efficacy of NanoVP in glioblastoma cells, wherein the intracellular VP concentration was determined via extraction method at 24 hours post-incubation with 0.25 μM NanoVP, free-form VP, or liposomal VP.

FIG. 11B shows the in vitro PDT efficacy of NanoVP in glioblastoma cells, wherein Cell viability was measured via MTT assay 24 hours after NanoVP, free-form VP, or Liposomal VP-mediated PDT (0-5 J/cm², 10 mW/cm²).

FIG. 11C shows the in vitro PDT efficacy of NanoVP in glioblastoma cells, where mitochondrial membrane potential depolarization was quantified via TMRE probe 1-hour after NanoVP, free-form VP, or Liposomal VP-mediated PDT (10 J/cm², 50 mW/cm²). Mitochondrial membrane potential depolarization was calculated using the formula: ΔΨm=1−(Tf/Tc), where Ta is the TMRE fluoresce signal from FCCP or a treatment group, and Tc is the TMRE fluoresce signal from the control group.

FIG. 11D shows the in vitro PDT efficacy of NanoVP in glioblastoma cells, wherein representative fluorescence images of the TMRE probe and phase constant images of U87 cells at 30 minutes after PDT.

FIG. 11E shows the in vitro PDT efficacy of NanoVP in glioblastoma cells, wherein the quantification of total and cleaved caspase 3 expressions in U87 cells at 1 hour post-PDT (10 J/cm², 50 mW/cm²). A two-tail (total) and one-tail (cleaved) t-test was used to calculate significant differences.

FIG. 11F shows the in vitro PDT efficacy of NanoVP in glioblastoma cells, wherein representative immunoblotting showed changes in total and cleaved caspase 3 expressions in U87 cells at 1-hour post-PDT.

FIG. 11G shows the in vitro PDT efficacy of NanoVP in glioblastoma cells, wherein cell viability was measured via MTT assay 72 hours after incubation with non-PDT activated (0 J/cm², 0 mW/cm²) NanoVP, free-form VP, or Liposomal VP.

FIG. 11H shows the in vitro PDT efficacy of NanoVP in glioblastoma cells, in terms of representative fluorescence images of NanoVP, free-form VP, or Liposomal VP within U87 cells. Scale bar=50 μm.

FIG. 11I shows the in vitro PDT efficacy of NanoVP in glioblastoma cells, in particular quantitative analyses of NanoVP, free-form VP, or Liposomal VP fluorescence signal in U87 cells. Fluorescence signals were normalized to the largest signal for each concentration. One-way ANOVA with multiple comparison test was used to calculate significant differences, where * P<0.05. ** P<0.01, and *** P<0.001. N≥3. Error bar shows the standard error of the mean.

FIG. 12A depicts the PDT efficacy within 3T3 fibroblast cells, wherein the intracellular VP concentration was determined via extraction methods at 24 hours post incubation with 0.25 μM of NanoVP, free-form VP, or Liposomal VP.

FIG. 12A shows the PDT efficacy within 3T3 fibroblast cells, wherein cell viability was measured via MTT assay 24 hours after NanoVP, free-form VP, or Liposomal VP-mediated PDT (0-5 J/cm², 10 mW/cm²). One-way ANOVA with multiple comparison test was used to calculate significant differences, where *** P<0.001. Error bars show standard errors of the mean.

FIG. 13A demonstrates that VP-mediated PEDT does not induce lysosomal damage. Lysosomal damages were assessed via NRU assay at 1-hour post-PDT J/cm², 50 mW/cm²) of 0.25 μM NanoVP, free-form VP, or Liposomal VP. One-way ANOVA with multiple comparison test was used to calculate significant differences, where * P<0.05, N≥3. Error bars show standard errors of the mean.

FIG. 13B demonstrates that VP-mediated PEDT does not induce lysosomal damage. Lysosomal damages were assessed via NRU assay at 24 hours post-PDT J/cm², 50 mW/cm²) of 0.25 μM NanoVP, free-form VP, or Liposomal VP. One-way ANOVA with multiple comparison test was used to calculate significant differences, where * P<0.05, N≥3. Error bars show standard errors of the mean.

FIG. 14A depicts how NanoVP is a substrate for ABC transported ABCG2 and P-gp. Intracellular VP concentration was determined via extraction methods at 4 hours post-incubation with 1 μM NanoVP or free VP. Breast cancer cells overexpressing P-gp (MCF-7 TX400) and parental MCF-7 cells were incubated with and without inhibitors (10 μM FTC for ABCG2 or 2.5 mM tariquidar for P-gp). Two-way ANOVA with multiple comparison test was used to calculate significant differences, where * P<0.05. ** P<0.01, N≥3. Error bar shows the standard error of the mean.

FIG. 14B depicts how NanoVP is a substrate for ABC transported ABCG2 and P-gp. Intracellular VP concentration was determined via extraction methods at 4 hours post-incubation with 1 μM NanoVP or free VP. Breast cancer cells overexpressing ABCG2 (MCF-7 MX100) and parental MCF-7 cells were incubated with and without inhibitors (10 μM FTC for ABCG2 or 2.5 mM tariquidar for P-gp). Two-way ANOVA with multiple comparison test was used to calculate significant differences, where * P<0.05, ** P<0.01, N≥3. Error bar shows the standard error of the mean.

FIG. 15 shows NanoVP dark toxicity in 3T3 cells. Cell viability was measured via MTT assay 72 hours after incubation with non-PDT activated (0 J/cm², 0 mW/cm²) NanoVP, free-form VP, or Liposomal VP.

FIG. 16A depicts the phototoxicity and biodistribution of NanoVP in U87 glioblastoma xenograft mouse model. PDT treatment (100 J/cm², 100 mW/cm²) was initiated ˜14 days after subcutaneous U87 cancer cell implantation when tumor volumes reached approximately 100 mm³. Mice were randomized into groups that received (i) no-treatment, (ii) Liposomal VP (0.5 mg/kg), (iii) NanoVP (0.5 mg/kg). FIG. 16A in particular shows how tumor volume was longitudinally monitored and calculated using the standard estimation formula, V=½×length×width², where length equals the maximum tumor diameter in millimeters and width equals the diameter that is perpendicular to the length. Tumor volumes were normalized to the initial volume at the time of treatment.

FIG. 16B shows the specific growth rate (SGR) of tumors 0-11 days and 11-25 days post-PDT were determined using the following formula: SGR=(1/V)(dV/dt), where V is tumor volume and t is time.

FIG. 16C is a Kaplan-Meier plot of tumor diameter greater than 1.5 cm (N=7-8 animals per group)

FIG. 16D shows the luantification of the surface area above the tumor that was impacted by PDT treatment.

FIG. 16E depicts representative digital images of tumors at 6 days post-PDT.

FIG. 16F shows the quantitative analyses of VP fluorescence intensity within the tumor and organs, 2 and 24 hours after VP injection. One-way ANOVA with multiple comparison test was used to calculate significant differences, where * P<0.05, ** P<0.01, and *** P<0.001. N≥3. Error bar shows the standard error of the mean.

FIG. 17 is a graph showing mouse weight post-PDT, wherein it is demonstrated that PDT had no impact on weight.

FIG. 18 shows VP biodistribution in mice organs and U87 tumors. This is conveyed through representative fluorescence images of NanoVP and liposomal VP in mice organs at 2 hours and 24 hours post-intravenous injection. Scale bar=3 mm.

FIG. 19A depicts PDT-induced BBB opening with the rat brain, and specifically shows a schematic depiction of experimental design. NanoVP (0.25 mg/kg) or 5-aminolevulinic acid (5-ALA, 20 mg/kg) were intravenously administered 30 minutes PDT (NanoVP: 690 nm, 80 J/cm²; 5-ALA: 635 nm, 80 J/cm²; 85 mW/cm²) was performed on the exposed right brain hemisphere. After 90 minutes, Evans blue was IV administered to the rats and circulated for 30 minutes before brain harvesting.

FIG. 19B depicts PDT-induced BBB opening with the rat brain, and specifically shows representative top and cross-sectional images of Evans blue within the brain after PDT-induced BBB opening.

FIG. 19C depicts PDT-induced BBB opening with the rat brain, and specifically shows the quantification of Evans blue extracted from the right brain hemispheres (N≥3 rats per group, background subtracted).

FIG. 19D depicts PDT-induced BBB opening with the rat brain, and specifically shows the quantification of maximum depth that extravasated Evans blue can be visualized within the brain.

FIG. 19E depicts PDT-induced BBB opening with the rat brain, and specifically shows representative histopathology of rat brain tissue after traditional and low-dose PDT. Photomicrographs of brain sections stained with Hematoxylin and cosin (H&E) and Luxol fast blue. Two tail t-test was used to calculate significant differences, * P<0.05. Error bar shows the standard error of the mean.

FIG. 20 contains a series of images showing how traditional PDT damages healthy brain tissue. In this Figure, representative photomicrographs of brain sections stained with H&E and Luxol fast blue. Photosensitizers (0.5 mg/kg VP or 125 mg/kg 5-ALA) were photoactivated (VP: 100 J/cm², 40 mW/cm²; 5-ALA: 60 J/cm², 40 mW/cm²) 30-minutes (for VP) or 4-hours (for 5-ALA) after IV injection. Brains were harvested 90 minutes post-PDT.

Various embodiments of the present disclosure will be described in detail regarding the drawings. Reference to various embodiments does not limit the scope of the disclosure. Figures represented herein are not limitations on the various embodiments according to the disclosure and are presented as an example illustration of the disclosure.

### DETAILED DESCRIPTION OF THE PREFERRED EMBODIMENTS

The present disclosure relates to facile surfactant-free compositions and methods of preparing amorphous nano drugs of verteporfin (NanoVP) with 100% active pharmaceutical ingredients and nearly 1,500-fold higher photosensitizer-loading capacity compared to standard Liposomal verteporfin. According to such methods and compositions, monodispersed NanoVP is self-quenched during storage in saline solution and can be de-quenched in cells, enabling PDT and fluorescence imaging of cancer. Beneficially, NanoVP enhances the anti-glioma PDT efficacy up to 10-fold in vitro and in vivo compared to Liposomal verteporfin. Importantly, as described in this disclosure, low doses of NanoVP-PDT can be used for safe, localized, and controlled blood-brain barrier opening, increasing drug accumulation in the brain by 5.5-fold compared to an FDA-approved photosensitizer (5-aminolevulinic acid-induced protoporphyrin IX). Collectively, the methods and compositions described herein represent a next-generation versatile photosensitizer formulation that can facilitate treatment strategies for brain tumors and invasive central nervous system diseases that are protected by the intact blood-brain barrier.

The embodiments of this disclosure are not limited to particular types of compositions or methods, which can vary. It is further to be understood that all terminology used herein is to describe particular embodiments only and is not intended to be limiting in any manner or scope. For example, as used in this specification and the appended claims, the singular forms “a,” “an” and “the” can include plural referents unless the context indicates otherwise. Unless indicated otherwise, “or” can mean any one alone or any combination thereof, e.g., “A, B, or C” means the same as any of A alone, B alone, C alone, “A and B,” “A and C,” “B and C” or “A, B, and C.” Further, all units, prefixes, and symbols may be denoted in their SI accepted form.

As used herein, the terms “comprise,” comprises,” comprising,” “include,” “includes,” and “including” can be interchanged and are to be construed as at least having the features to which they refer while not excluding any additional unspecified features.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, a description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6, and decimals and fractions, for example, 1.2, 3.8, 1½, and 4¾ This applies regardless of the breadth of the range.

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

The terms “a,” “an,” and “the” include both singular and plural referents.

The term “or” is synonymous with “and/or” and means any one member or combination of members of a particular list.

The term “about,” as used herein, refers to variation in the numerical quantity that can occur, for example, through typical measuring techniques and equipment, with respect to any quantifiable variable, including, but not limited to, mass, volume, time, temperature, pH, reflectance, whiteness, etc. Further, in practical handling procedures, there is certain inadvertent error and variation that is likely through differences in the manufacture, source, or purity of the ingredients used to make the compositions or carry out the methods and the like. The term “about” also encompasses amounts that differ due to different equilibrium conditions for a composition resulting from a particular initial mixture. The term “about” also encompasses these variations. Whether or not modified by the term “about,” the claims include equivalents to the quantities.

As used herein, a “photosensitizer” or “photoreactive agent” is a compound or composition that is useful in photodynamic therapy in that it absorbs electromagnetic radiation and emits energy sufficient to exert a therapeutic effect, e.g., the impairment or destruction of unwanted cells or tissue, or sufficient to be detected in diagnostic applications. Photodynamic therapy according to the invention can be performed using any of a number of photoactive compounds. For example, the photosensitizer can be any chemical compound that collects in one or more types of selected target tissues and, when exposed to the light of a particular wavelength, absorbs the light and induces impairment or destruction of the target tissues. Virtually any chemical compound that homes to a selected target and absorbs light may be used in this invention. Preferably, the photosensitizer is nontoxic to the patient to which it is administered and is capable of being formulated in a nontoxic composition. The photosensitizer is also preferably nontoxic in its photodegraded form. Ideal photosensitizers are characterized by a lack of toxicity to cells in the absence of the photochemical effect and are readily cleared from non-target tissues.

“Theoretical loading capacity” and “loading capacity” herein refer to the amount of photosensitizers loaded per unit weight of the nanoparticle(s), with the former encompassing the calculated loading capacity and the former the empirical loading capacity. The nanoparticles described herein may have a loading capacity and/or theoretical loading capacity of up to 100% by weight, including 50 wt. %, 60 wt. %, 70 wt %, 80 wt. %, 90 wt. % and all integers included within these ranges. For example, the loading capacity of the nanoparticle compositions described herein is up to 100% by weight (i.e., 100 wt. %) or is 100 wt. % with, for example, 706,000±38,000 verteporfin molecules per nanoparticle. A 100% loading capacity beneficially permits carrier-free and/or lipid-free compositions and delivery of the same. In comparison, most of the existing nanomedicines possess the drawback of low drug-loading capacity (generally less than 10%) associated with more carrier materials.

The methods, systems, apparatuses, and compositions disclosed herein may comprise, consist essentially of, or consist of the components and ingredients described herein as well as other ingredients not described herein. As used herein, “consisting essentially of” means that the methods, systems, apparatuses, and compositions may include additional steps, components, or ingredients, but only if the additional steps, components, or ingredients do not materially alter the basic and novel characteristics of the claimed methods, systems, apparatuses, and compositions.

It should also be noted that, as used in this specification and the appended claims, the term “configured” describes a system, apparatus, or other structure that is constructed or configured to perform a particular task or adopt a particular configuration. The term “configured” can be used interchangeably with other similar phrases such as arranged and configured, constructed and arranged, adapted and configured, adapted, constructed, manufactured and arranged, and the like.

The “scope” of the present disclosure is defined by the claims, along with the full scope of equivalents to which such claims are entitled. The scope of the disclosure is further qualified as including any possible modification to any of the aspects and/or embodiments disclosed herein which would result in other embodiments, combinations, sub-combinations, or the like that would be obvious to those skilled in the art.

### Discussion of the Technology

PDT using Liposomal VP has already helped hundreds of thousands of patients globally with wet age-related macular degeneration. Since its FDA approval in 2001, Liposomal VP has been tested clinically to treat a wide range of cancers, including primary breast, retinoblastoma, and refractory brain tumors. Liposomal VP-PDT is currently being evaluated for unresectable solid pancreatic tumors or advanced pancreatic cancer (NCT03033225), and the chemotherapeutic effects of Liposomal VP are being evaluated in patients with recurrent high-grade EGFR-mutated glioblastoma (NCT04590664). Free-form VP is not used clinically because the large VP agglomerations created in aqueous buffers will negatively impact its pharmacokinetics and singlet oxygen yield. Pure drug nanoparticle delivery systems offer tools to improve the pharmacokinetic profile of hydrophobic drugs and minimize the reliance on solubilizing agents.

The present disclosure provides a novel pure drug nanoparticle of VP (NanoVP) that significantly improves photosensitizer delivery to cancer cells. The in vivo examples provided herein show the safety, feasibility, and potential utility of NanoVP for PDT of gliomas, as well as the blood-brain barrier (BBB) opening to enhance drug delivery with no evidence of microscopic normal tissue injury. Solvent-antisolvent precipitation is a simple and reproducible formulation strategy to synthesize stable nanoparticles of VP. The size of NanoVP is tunable between 65 and 150 nm by increasing the initial photosensitizer concentration in solvent or the DMSO: Water ratio. Similarly, others demonstrated a larger initial concentration and higher solvent: antisolvent ratio could increase the size of nano drug paclitaxel.

Synthesis optimization revealed that monodispersed NanoVP can be produced using an initial VP concentration of less than 15 mM and a DMSO: Water ratio below 6%. A future direction includes examining the impact of solvent type (e.g., ethanol or methanol) on NanoVP formation, microstructure, size, and stability. The amorphous structure of the prepared NanoVP, shown by electron diffraction data, represents another major advantage in the quest to immediately convert quenched NanoVP into its unquenched molecular form in the presence of the serum components or cells, which recovers fluorescence signal and PDT capability.

Previous studies have also shown that pure drug nanoparticles can improve cellular pharmacokinetics while liposomes hinder therapeutic uptake. As a direct result of improved cellular accumulation. NanoVP was found to be a more potent PDT agent than the traditional Liposomal VP. This result is similar to previous studies that have shown free-form drugs, including doxorubicin and paclitaxel, are uptaken more effectively in various cancer cells compared to their liposomal counterparts. Free-form VP is lipophilic and can passively diffuse through the cell membrane. It is speculated that NanoVP had a similar accumulation rate as free-form VP, as NanoVP is readily dissociated into its molecular form in the presence of the serum components or cells. When VP is encapsulated within a liposome, the mode of cellular entry is generally altered from passive diffusion to endocytosis potentially hindering the overall drug accumulation rate. The present disclosure demonstrates that NanoVP-PDT, similar to free-form VP-PDT, effectively produces intracellular ROS, induces mitochondrial membrane potential (ΔΨm) depolarization, and initiates intrinsic apoptosis upon light activation. Typically, PDT requires very low doses of photosensitizers (in the nanomolar to the micromolar range) to be effective. Recent clinical and preclinical studies have reported high concentrations of VP (in the millimolar range) can induce ‘dark’ cytotoxicity in GBM cells. A 10-100-fold higher dose of VP, compared to the PDT dose, has been shown to downregulate Bcl-2, disrupt the YAP/TAZ-TEAD complex interaction, and induce cancer cell death without light activation. The present disclosure further demonstrates that the enhanced cellular uptake of NanoVP resulted in superior non-PDT (‘dark’) killing effects. Alternatively, Liposomal VP alone did not inhibit cancer growth at the same millimolar incubation concentrations.

Like chemotherapy, radiation, and immunotherapy. NanoVP-PDT would require multiple treatment cycles or combination regimens to effectively eradicate the entire cancerous cell population and warrants further investigation. Nonetheless, a single cycle of NanoVP-PDT remains an attractive adjuvant therapy that can be combined with surgery. In prior clinical evaluation, Liposomal VP-PDT decreasing tumor volume could convert patients with previously unresectable pancreatic tumors to become candidates for surgery. In addition to surgery, PDT has been successfully combined with chemotherapy, radiation, or immunotherapy due to different mechanisms of action and their non-overlapping side effects. Biodistribution analysis reveals NanoVP tended to have a higher tumor accumulation, while Liposomal VP accumulation increased within the liver, pancreas, and spleen. Overall, NanoVP can be an adequate replacement for the Liposomal VP formulation.

Drug delivery to brain tumors remains especially difficult due to the BBB, a specialized network of capillaries in the brain. The high degree of tightness and integrity imposed by the BBB limits the effective delivery of more than 98% of small-molecule therapeutics to the brain. While many treatment modalities have shown promise in the opening (i.e., permeabilizing) the BBB and enhancing brain drug delivery, they often result in edema and neurotoxicity, limiting their clinical use. Numerous groups have shown the potential of light-activated 5-ALA (GliolanR)-induced protoporphyrin IX (PpIX) to open the BBB in rodents. Hirschberg and colleagues showed larger doses of 5-ALA-PDT could open the BBB for up to 72 hours, but treatment resulted in early signs of necrosis up to 5 mm away from the primary brain tumor. In addition, non-tumor-bearing animals that received 125 mg/kg 5-ALA and 54 J experienced a 50% mortality within 5 days of treatment. The present disclosure demonstrates that NanoVP-PDT mediates BBB opening to increase EB accumulation in the brain by 5.5-fold compared to using 5-ALA-PDT. More importantly, the present disclosure demonstrates for the first time that low-dose PDT using NanoVP did not result in healthy brain tissue damage.

Finally, intraoperative NanoVP-PDT has a high translational potential. As described herein, despite promising results, the side effects of PDT remain a major concern. An initial application of low-dose PDT following tumor resection provides a safe and advantageous treatment regimen. In this context, patients may receive PDT intraoperatively via fiber optic light conduits placed within the resection cavity. Relatedly, the methods disclosed herein may be used to open the BBB and sterilize unresectable, brain-invading tumor cells in real-time, under controlled conditions, during open surgery. In such a case, light penetration depth (defined as the depth at which the incident optical energy drops to 37%, 1/e) is preferably 1-4 mm in most tissues, although diffused light (i.e., the remaining 37% optical energy) could reach up to 1.5-2 cm in tissues. This penetration depth enables VP-PDT to manage post-surgical residual GBM cells (typically within 1-2 cm from the border of the original lesion) that are responsible for ˜80% recurrence. Moreover, the self-limiting depth of effect avoids non-specific priming of the underlying tissue. Rarely, the GBM recurrence occurs distant beyond 2 cm of the resection cavity. For distant recurrence (“ghost” cells) or inoperable GBM, light can be delivered to the entire tumor using stereotactically placed fibers in the clinic.

### Compositions

### Photosensitizer

The compositions preferably comprise one or more photosensitizers, which are compounds that absorb light energy. The photosensitizer can absorb light from about 400 nm to about 1000 nm, inclusive of all integers within this range (e.g., 400 nm, 401 nm, 402 nm, etc.). In an embodiment, the photosensitizer has low solubility and high permeability, or low solubility and low permeability.

The photosensitizer can be any composition that absorbs light and initiates a photochemical reaction that produces cytotoxic products. For example, suitable photosensitizers that can be used include, but are not limited to, haematoporphyrins, photofrins, chlorins such as meta-tetra hydroxyphenyl chlorin, mono-L-aspartyl chlorin e6, or bacteriochlorins, or derivatives thereof. The photosensitizer can also include phthalocyanines, porphyrins, benzoporphyrins, 5-aminolevulinic acid (ALA), or derivatives thereof. Other photosensitizers include, but are not limited to, purpurins, porphycenes, pheophorbides, and verdins. Purpurins are a class of porphyrin macrocycle with an absorption band at from about 630 nm to about 715 nm, typified by tin etiopurpurin (SnET2), which has an extinction coefficient of 40,000 M⁻¹ cm⁻¹ at about 700 nm. Porphycenes, having activation wavelengths of about 635 nm, are also useful. Phorbides are derived from chlorophylls (e.g. pheophorbide) and are also useful as photosensitizers. Verdins contain a cyclohexanone ring fused to one of the pyrroles of the porphyrin ring and can also be used as a photosensitizer. Psoralens are another example of a photosensitizer that can be used in the disclosed conjugates and methods.

In a preferred embodiment, the photosensitizer comprises a porphyrin. Suitable porphyrin photosensitizers include but are not limited to, hematoporphyrin and derivatives thereof (HpD), photofrin, verteporfin, or a combination thereof. Further discussion of porphyrin photosensitizers is found in Kou et al., Porphyrin photosensitizers in photodynamic therapy and its applications, ONCOTARGET, 2017; 8(46): 81591-81603, which is herein incorporated by reference in its entirety. In a preferred embodiment, the photosensitizer is free-form verteporfin (VP) powder.

In an embodiment, the photosensitizer comprises a second-generation photosensitizer designed to meet a specific demand, such as a benzoporphyrin derivative monoacid ring A (BPD-MA) verteporfin, meso-tetrakis (4-sulfonatophenyl) porphyrin (TPPS), N-aspartyl chlorin e6 NPe6, aminolevulinic acid (5-ALA), temoporfin or m-THPC, TSPP, HPPH, hypericin, or a combination thereof. Alternatively, the photosensitizer may comprise a third-generation photosensitizer such as gold-nanoclustered hyaluronan nano-assemblies, chlorin E6 (Ce6)+upconversion nanoparticles, photofrin+gap junctional intercellular communication, Ce6+tumor-targeting photosensitizer, Ce6+chitoUDCA nanoparticles, ICG-loaded nanospheres, including those coated with chitosan, or a combination thereof.

Synthetic non-porphyrin compounds can also be used as photosensitizers in the compositions and methods disclosed herein. Suitable non-porphyrin compounds include, but are not limited to, phenothiazinium compounds such as methylene blue, Toluidine blue, a cyanine such as Merocyanine-540, an acridine dye, Nile blue, and/or rhodamine such as the mitochondria-specific Rhodarnine 123.$The photosensitizer may also comprise a benzoporphyrin derivative, such as a benzoporphyrin mono acid derivative.

The one or more photosensitizers may be present in an initial (i.e., pre-formation into nano structure) concentration of between about 100 micromolar (μM) to about 100 millimolar (mM), between about 1 mM to about 50 mM, or between about 5 mM to about 10 mM, inclusive of all integers within these ranges. In a preferred embodiment, the initial concentration of the photosensitizer is about 7 mM.

The one or more photosensitizers may be present in a final (i.e., post-formation into nano structure) concentration of up to 100 wt. %. In an embodiment, the photosensitizers described herein preferably comprise 100 wt. % of the amorphous nanoparticle formulation.

### Solvent

The photosensitizer is preferably combined with a solvent, preferably an organic solvent in which the photosensitizer is soluble. More particularly, the selected solvent is preferably a water-miscible solvent and/or a solvent capable of dissolving the photosensitizer to a greater extent so that a clear solution is obtained. Such solvents include but are not limited to water-miscible protic compounds, in which a hydrogen atom in the molecule is bound to an electronegative atom such as oxygen, nitrogen, or other Group VA, VIA, and VII A in the Periodic Table of elements. Examples of such solvents include, but are not limited to, alcohols, amines (primary or secondary), oximes, hydroxamic acids, carboxylic acids, sulfonic acids, phosphonic acids, phosphoric acids, amides, and urea.

Other examples of the solvent also include aprotic organic solvents. Some of these aprotic solvents can form hydrogen bonds with water but can only act as proton acceptors because they lack effective proton donating groups. One class of aprotic solvents is a dipolar aprotic solvent, which is a solvent with a comparatively high relative permittivity (or dielectric constant) and a sizable permanent dipole moment that cannot donate suitably labile hydrogen atoms to form strong hydrogen bonds, e.g. dimethyl sulfoxide (DMSO).

Dipolar aprotic solvents can include, for example, amides, ureas, ethers, cyclic ethers, nitriles, ketones, sulfones, sulfoxides, fully substituted phosphates, phosphonate esters, phosphoramides, nitro compounds, and the like. Dimethylsulfoxide (DMSO), N-methyl-2-pyrrolidinone (NMP), 2-pyrrolidinone, 1,3-dimethylimidazolidinone (DMI), dimethylacetamide (DMA), dimethylformamide (DMF), dioxane, acetone, tetrahydrofuran (THF), tetramethylenesulfone (sulfolane), acetonitrile, and hexamethylphosphoramide (HMPA), nitromethane, among others, are similarly suitable.

Particularly preferred solvents comprise dimethyl sulfoxide (DMSO), acetone, ethanol, DCM, methanol, NMP, CAN, or a combination thereof.

### Antisolvent

The photosensitizer and solvent combination are preferably combined with an antisolvent, specifically a solvent in which the photosensitizer is least soluble or completely insoluble. Accordingly, the photosensitizer is preferably not soluble in the antisolvent, and/or the solvent is soluble in the antisolvent. At a minimum, the solvent is preferably more soluble in the antisolvent than the photosensitizer in order to induce precipitation/crystallization.

Preferably, the antisolvent is an aqueous solvent. This aqueous solvent may be water by itself. This solvent may also contain buffers, salts, surfactant(s), water-soluble polymers, and combinations of these excipients. Examples of suitable antisolvents include, without limitation, deionized water, PBS, or a combination thereof.

### Solvent-Antisolvent Ratio

The solvent and antisolvent may be provided in any suitable ratio. In a preferred embodiment, the solvent-to-antisolvent ratio is between about 1:100 to about 1:3, between about 1:80 to about 1:20, or between about 1:50 to about 1:30. In a preferred embodiment, the solvent-antisolvent ratio is about 1:40.

### Amorphous Nanoparticles

The nanoparticles described herein are preferably provided in an amorphous form. As demonstrated in a series of in chemical, in vitro, and in vivo assays, the amorphous nanoparticle form of the photosensitizer (particularly verteporfin) is superior to its clinically relevant liposomal formulation, other clinically used porphyrins, and comparable uniform crystalline formations. In particular, the amorphous nanoparticle formulation significantly improved the accumulation of the intracellular photosensitizer, the tumor-to-normal tissue ratio of photosensitizers, and the anti-cancer efficacy of photodynamic therapy in human brain cancer cells and xenograft mouse models, compared to the liposomal formulation. In addition, low-energy light activation of the amorphous nanoparticle formulation of verteporfin selectively and safely improved the blood-brain barrier permeability to model drugs in rats, compared to the clinically approved photosensitizer-5-aminolevulinic acid-induced protoporphyrin IX.

The size of the amorphous nanoparticles can be tuned by adjusting the photosensitizer concentration in the solvent. In some examples, a pharmaceutically acceptable solubilizing agent is not needed for the preparation of the amorphous nanoparticles, but at least one pharmaceutically acceptable solubilizing agent can be added. The nanoparticles described herein have a size of preferably about 1000 nm or less, about 200 nm or less, or about 180 nm or less. Still more preferably the nanoparticles have a size of between about 40 nm and about 180 nm, or between about 40 nm and 170 nm.

As described herein, the formation of the amorphous nanoparticles can be induced by adding the solvent/photosensitizer solution dropwise into the antisolvent. A nanosize dispersion is generated through an energy addition step of adding energy through any suitable means, such as stirring, sonication, homogenization, countercurrent flow homogenization, microfluidization, or other methods of providing impact, shear, or cavitation forces.

The amorphous nanoparticles further demonstrate excellent stability over time. In an embodiment, the amorphous nanoparticles and compositions comprising the same are shelf stable for long periods of time, for example, up to 1 month, up to 6 months, or up to 1 year or more. Polymers

In an embodiment, the presently disclosed compositions may be synthesized with a polymer to form NanoVP-coated polymers. In a preferred embodiment, the polymer is a neutral polymer or a cationic polymer.

A suitable polymer is polyethylene glycol (PEG), including cationic PEG-lipid (CPL) conjugates). The molecular weights of the PEG can vary, for example, from 200 to 50,000. Some commonly used PEGs that are commercially available include PEG 350, PEG 550, PEG 750, PEG 1000, PEG 2000, PEG 3000, and PEG 5000. The phospholipid or the PEG-phospholipid conjugate may also incorporate a functional group that can covalently attach to a ligand including but not limited to proteins, peptides, carbohydrates, glycoproteins, antibodies, or pharmaceutically active agents. These functional groups may conjugate with the ligands through, for example, amide bond formation, disulfide or thioether formation, or biotin/streptavidin binding. Examples of the ligand-binding functional groups include but are not limited to hexanoylamine, dodecanylamine, 1,12-dodecanedicarboxylate, thioethanol, 4-(p-maleimidophenyl) butyramide (MPB), 4-(p-maleimidomethyl)cyclohexane-carboxamide (MCC), 3-(2-pyridyldithio) propionate (PDP), succinate, glutarate, dodecanoate, and biotin.

In an embodiment, the cationic polymer comprises polyethyleneimine (PEI). In a preferred embodiment, the neutral polymer comprises poloxamer 407.

In an embodiment, the nanoparticles described herein are formed without requiring lipids or polymers. Relatedly, in an embodiment, the nanoparticles are free of lipids and polymers.

### ABC Transporters

In an embodiment, the nanoparticles disclosed herein may be used as a substrate for ABC transporters. ABC transporters classified can be generally grouped into exporters and importers with the importers further divided into two classes (I and II), depending on the details of their architecture and mechanism. Key structural features of ABC transporters include (1) an outward-facing maltose transporter with ADP·VO₄ in catalytic sites and maltose bound to the transmembrane domain (TMD), (2) a homodimeric exporter Sav1866 from Staphylococcus aureus in the outward-facing conformation with ADP in catalytic sites, (3) P-glycoprotein in the inward-facing conformation with an inhibitor molecule bound at the TMDs, (4) the nucleotide-binding domain (NBD) sandwich dimer of the maltose transporter (MalK) as seen from the cytoplasmic side, (5) the cavity formed by the TMDs of outward-facing Sav 1866, and (6) a cross-section through the TMDs of glycoprotein showing the two inhibitor molecules.

More particularly, there are 48 ABC transporters in humans that can be divided into seven sub-families A through G. Mammalian ABC transporters are involved in the cellular export of several groups of molecules, including cholesterol and sterols, lipids, retinoic acid derivatives, bile acid, iron, nucleosides, and peptides. These transporters have been observed in several genetic conditions, including Tangier (ABCA1) and Stargardt (ABCA4) disease, immune deficiency and cancer (ABCB⅔; TAP transporter), cystic fibrosis (cystic fibrosis transmembrane conductance regulator [CFTR]; ABCC7), and adrenoleukodystrophy (ABCD1), among others. Another prominent group of human ABC transporters is found in the liver, placenta, and blood-brain barrier where they are involved in the detoxification of hydrophobic organic molecules. The group includes P-glycoprotein (P-gp, ABCB1), MRP (ABCC1), and ABCG2. These transporters, when found highly expressed in the plasma membrane of tumor cells, can significantly impact tumor cell death and disease prognosis.

In an embodiment where the nanoparticles disclosed herein are used as a substrate for an ABC transport, the transporter comprises an ABC transporter found in the blood-brain barrier. In a preferred embodiment, the ABC transporter comprises ABCG2, P-gp, or a combination thereof.

### Conjugates

The nanostructures described herein may optionally be provided as part of a conjugate, wherein the conjugate is a combination of the one or more photosensitizers and a conjugating agent. The conjugate agent may, for example, bind to the photosensitizer to facilitate delivery of the photosensitizer to a desired location.

In an embodiment, the conjugating agent may comprise an antibody that specifically binds an antigen in a manner sufficient to deliver a photosensitizer to the desired location. The antibody may include, without limitation, a full-length antigen or fragment thereof. A polypeptide to be used for generating an antibody of the disclosed conjugates and methods can be partially or fully purified from a natural source or can be produced using recombinant DNA techniques. Accordingly, the antibody may comprise a monoclonal antibody, a human antibody, a genetically manipulated human antibody, a non-human antibody, or a combination thereof. Further discussion of photosensitizer-antibody coupling is found in U.S. Patent Pub. No. 2006/0231107, which is herein incorporated by reference in its entirety.

### Additional Optional Ingredients

The compositions described herein may optionally comprise one or more additional ingredients, such as excipients, disintegrants, lubricants, penetration enhancers, pH modifiers, pH buffers, surfactants, or a combination thereof, to facilitate clinical use and administration.

Disintegrants that can be used include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, com oil, and soybean oil), zinc stearate, ethyl oleate, ethyl laureate, agar, and mixtures thereof. Additional lubricants include, for example, a syloid silica gel, a coagulated aerosol of synthetic silica, a pyrogenic silicon dioxide, or a combination thereof.

Further, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired.

Depending on the specific tissue to be treated, one or more penetration enhancers can be used to assist in delivering the nanoparticles to the target location. Suitable penetration enhancers include, but are not limited to acetone, an alcohol (for example ethanol, oleyl, and tetrahydro furyl) alkyl sulfoxide (such as dimethyl sulfoxide), dimethyl acetamide, dimethyl formamide, polyethylene glycol (PEG), a pyrrolidone (such as polyvinylpyrrolidone), povidone, polyvidone, urea, and various water-soluble or insoluble sugar esters such as polysorbate (e.g., Tween 80) or sorbitan monostearate (e.g., Span 60).

One or more zwitterionic surfactants may be optionally incorporated into the compositions described herein. Zwitterionic surfactants are electrically neutral but possess local positive and negative charges within the same molecule. Suitable zwitterionic surfactants include but are not limited to zwitterionic phospholipids, such as phosphatidylcholine, phosphatidylethanolamine, diacyl-glycero-phosphoethanolamine (such as dimyristoyl-glycero-phosphoethanolamine (DMPE), dipalmitoyl-glycero-phosphoethanolamine (DPPE), distearoyl-glycero-phosphoethanolamine (DSPE), and dioleolyl-glycero-phosphoethanolamine (DOPE).

Suitable cationic surfactants include but are not limited to quaternary ammonium compounds, such as benzalkonium chloride, cetyltrimethylammonium bromide, chitosans, lauryldimethylbenzylammonium chloride, acyl carnitine hydrochlorides, dimethyldioctadecylammonium bromide (DDAB), dioleoyltrimethylammonium propane (DOTAP, also known as N-[1-(2,3-dioleoyloxy) propyl]-N, N, N-trimethylammonium), N-[1-(2,3-dioleyloxy)propyl]-N, N, N-trimethylammonium (DOTMA), dimyristoyltrimethylammonium propane (DMTAP), dimethylaminoethanecarbamoyl cholesterol (DC-Chol), 1,2-diacylglycero-3-(O-alkyl)phosphocholine, O-alkylphosphatidylcholine, alkyl pyridinium halides, or long-chain alkyl amines such as, for example, n-octylamine and oleylamine.

Suitable nonionic surfactants include glyceryl esters, polyoxyethylene fatty alcohol ethers (MACROGOL™ and BRIJ™), polyoxyethylene sorbitan fatty acid esters (polysorbates), polyoxyethylene fatty acid esters (MYRJ™), sorbitan esters (SPAN™), glycerol monostearate, polyethylene glycols, polypropylene glycols, cetyl alcohol, cetostearyl alcohol, stearyl alcohol, aryl alkyl polyether alcohols, polyoxyethylene-polyoxypropylene copolymers (poloxamers), poloxamines, methylcellulose, hydroxymethylcellulose, hydroxypropylcellulose, hydroxypropylmethylcellulose, noncrystalline cellulose, polysaccharides including starch and starch derivatives such as hydroxyethyl starch (HES), polyvinyl alcohol, and polyvinylpyrrolidone. The nonionic surfactant may also comprise a polyoxyethylene and polyoxypropylene copolymer (e.g., those according to the structure EO/PO/EO, PO/EO/PO, and EO/PO).

### Methods of Forming the Nanoparticles

In an embodiment, the nanoparticles described herein are preferably formed through solvent-antisolvent precipitation techniques. This process generally includes the steps of (1) preparing a liquid phase of a photosensitizer in a solvent or a combination of solvents in which the photosensitizer is soluble and to which may be optionally added one or more surfactants; (2) preparing a second liquid phase of an antisolvent or a combination of antisolvents, wherein the antisolvent is miscible with the solvent for the photosensitizer; (3) adding the solution of (1) dropwise into the solution of (2), preferably in the presence of agitation, e.g. by stirring or sonication; and (4) removing any remaining solvent, antisolvent, and a photosensitizer to produce crystallized nanoparticles.

Any other suitable precipitation method may be used, including without limitation microprecipitation methods, emulsion precipitation, phase inversion precipitation, pH shift precipitation, infusion precipitation, temperature shift precipitation, solvent evaporation precipitation, reaction precipitation, compressed fluid precipitation, nanosphere/microsphere precipitation, or a combination thereof. Further discussion of methods of precipitation can be found in U.S. Pat. No. 10,952,965, which is herein incorporated by reference in its entirety.

### Methods of Administration

The method of administration will vary depending on the particular use, e.g., whether as part of cancer therapy, drug delivery, imaging, etc. Single unit dosage forms of the compositions described herein are suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: a tablet, caplet, capsule, such as a soft elastic gelatin capsule or softgel, tablet, capsule, spansule, liquid, granula, powder, cachet, troche, lozenge, dispersion, suppository, ointment, cataplasm (poultice), paste, powder, dressing, cream, plaster, solution, patch, aerosol (e.g., nasal spray or inhaler), gel, liquid dosage form suitable for oral or mucosal administration to a patient, including a suspension (e.g., aqueous or non-aqueous liquid suspension, oil-in-water emulsion, or water-in-oil liquid emulsion), solution, and elixir, liquid dosage forms suitable for parenteral administration to a patient, a sterile solid (e.g., a crystalline or amorphous solid) that can be reconstituted to provide liquid dosage forms suitable for parenteral administration, or any combination thereof.

Similarly, the composition, shape, and type of dosage forms of the disclosure will typically vary depending on their use. For example, a dosage form used in the acute treatment of inflammation or a related disorder may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease or disorder.

The light source used to activate the photosensitizer in the nanoparticles can be from any suitable light source. The light can be in the form of a laser or in the form of a fiber optic source used to deliver light to the treatment site from a laser.

### Methods of Use in PDT for Cancer Therapy

In an embodiment, the nanoparticles described herein are used as an antineoplastic drug, and specifically as a chemotherapeutic agent. Alternatively or additionally, the nanoparticles described herein may be used as part of imaging and tumor identification, particularly fluorescence-guided resection of tumors.

Cancer is characterized primarily by an increase in the number of abnormal cells derived from a given normal tissue, invasion of adjacent tissues by these abnormal cells, or lymphatic or blood-borne spread of malignant cells to regional lymph nodes and to distant sites (metastasis). Current cancer therapy may involve surgery, chemotherapy, hormonal therapy, radiation treatment, biological therapy, and/or immunotherapy to eradicate neoplastic cells. The nanoparticles and compositions described herein may be used as part of cancer therapy, wherein the cancer includes, without limitation, peritoneal cancer, liver cancer, pancreatic cancer, brain cancer (e.g., glioblastoma treatment), neck cancer, spinal cancer, lung cancer, prostate cancer, bladder cancer, skin cancer, eye cancer, oral cancer, head and neck cancer, breast cancer, blood cancer, bone cancer, stomach cancer, kidney cancer, colorectal cancer, cervical cancer, ovarian cancer, central nervous system tumor, or a combination thereof.

Provided herein are methods of treating a disease, particularly cancer through PDT, comprising (a) administering to a target location a plurality of amorphous nanoparticles comprising a photosensitizer in an amount effective to facilitate photodynamic therapy (PDT) and (b) exposing the amorphous nanoparticles to photoactivating light having a wavelength capable of being absorbed by the photosensitizer; thereby (c) producing cytotoxic reactive oxygen species at the target location. In an embodiment, the target location is a tumor, a tissue, a cell, a vessel, or a combination thereof. Examples of irradiation devices and methods of using the same can be found in U.S. Pat. No. 9,974,974, which is herein incorporated by reference in its entirety.

Also provided herein are methods of treating a disease, particularly cancer, through the use of a chemotherapeutic agent, the method comprising administering to a target an effective amount of a composition comprising amorphous nanoparticles comprising a chemotherapeutic agent (e.g., verteporfin). In an embodiment, the composition further comprises or is administered in conjunction with an antineoplastic drug or a secondary therapeutic. In an embodiment, the target is located in a mammal, particularly a human. In an embodiment. the target comprises a tumor, a tissue, a cell, a vessel, or a combination thereof.

The nanoparticles described herein may be administered in conjunction with an antineoplastic drug comprising, for example, cmaxanib, cyclosporin, etanercept, doxycycline, bortezomib, acivicin, aclarubicin, acodazole hydrochloride, acronine, adozelesin, aldesleukin, altretamine, ambomycin, ametantrone acetate, amsacrine, anastrozole, anthramycin, asparaginasc, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, celecoxib, chlorambucil, cirolemycin, cisplatin, cladribine, crisnatol mesylate, cyclophosphamide, cytarabine, dacarbazine, dactinomycin, daunorubicin hydrochloride, decitabine, dexormaplatin, dezaguanine, dezaguanine mesylate, diaziquone, docetaxel, doxorubicin, doxorubicin hydrochloride, droloxifene, droloxifene citrate, dromostanolone propionate, duazomycin, edatrexate, eflomithine hydrochloride, elsamitrucin, enloplatin, enpromate, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, flurocitabine, fosquidone, fostriecin sodium, gemcitabine, gemcitabine hydrochloride, hydroxyurea, idarubicin hydrochloride, ifosfamide, ilmofosine, iproplatin, irinotecan, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitogillin, mitomalcin, mitomycin, mitosper, mitotane, mitoxantrone hydrochloride, mycophenolic acid, nocodazole, nogalamycin, ormaplatin, oxisuran, paclitaxel, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, riboprine, safingol, safingol hydrochloride, semustine, simtrazene, sparfosate sodium, sparsomycin, spirogermanium hydrochloride, spiromustine, spiroplatin, streptonigrin, streptozocin, sulofenur, talisomycin, tecogalan sodium, taxotere, tegafur, teloxantrone hydrochloride, temoporfin, teniposide, teroxirone, testolactone, thiamiprine, thioguanine, thiotepa, tiazofurin, tirapazamine, toremifene citrate, trestolone acetate, triciribine phosphate, trimetrexate, trimetrexate glucuronate, triptorelin, tubulozole hydrochloride, uracil mustard, uredepa, vapreotide, vinblastine sulfate, vincristine sulfate, vindesine, vindesine sulfate, vinepidine sulfate, vinglycinate sulfate, vinleurosine sulfate, vinorelbine tartrate, vinrosidine sulfate, vinzolidine sulfate, vorozole, zeniplatin, zinostatin, zorubicin hydrochloride, or a combination thereof.

Alternatively, the nanoparticles described herein may be administered in combination with a secondary therapeutic comprising, for example, 20-epi-1,25 dihydroxyvitamin D3, 5-ethynyluracil, abiraterone, aclarubicin, acylfulvene, adecypenol, adozelesin, aldesleukin, ALL-TK antagonists, altretamine, ambamustine, amidox, amifostine, aminolevulinic acid, amrubicin, amsacrine, anagrelide, anastrozole, andrographolide, angiogenesis inhibitors, antagonist D, antagonist G, antarelix, anti-dorsalizing morphogenetic protein-1, antiandrogen, prostatic carcinoma, antiestrogen, antineoplaston, antisense oligonucleotides, aphidicolin glycinate, apoptosis gene modulators, apoptosis regulators, apurinic acid, ara-CDP-DL-PTBA, arginine deaminase, asulacrine, atamestane, atrimustine, axinastatin 1, axinastatin 2, axinastatin 3, azasetron, azatoxin, azatyrosine, baccatin III derivatives, balanol, batimastat, BCR/ABL antagonists, benzochlorins, benzoylstaurosporine, beta lactam derivatives, beta-alethine, betaclamycin B, betulinic acid, bFGF inhibitor, bicalutamide, bisantrene, bisaziridinylspermine, bisnafide, bistratene A, bizelesin, breflate, bropirimine, budotitane, buthionine sulfoximine, calcipotriol, calphostin C, camptothecin derivatives, capecitabine, carboxamide-amino-triazole, carboxyamidotriazole, CaRest M3, CARN 700, cartilage derived inhibitor, carzelesin, casein kinase inhibitors (ICOS), castanosperminc, cecropin B, cetrorelix, chlorins, chloroquinoxaline sulfonamide, cicaprost, cis-porphyrin, cladribine, clomifene analogues, clotrimazole, collismycin A, collismycin B, combretastatin A4, combretastatin analogue, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, decitabine, dehydrodidemnin B, deslorelin, dexamethasone, dexifosfamide, dexrazoxane, dexverapamil, diaziquone, didemnin B, didox, diethylnorsperminc, dihydro-5-azacytidine, dihydrotaxol, 9-, dioxamycin, diphenyl spiromustine, docetaxel, docosanol, dolasetron, doxifluridine, doxorubicin, droloxifene, dronabinol, duocarmycin SA, ebselen, ecomustine, edelfosine, edrecolomab, eflomithine, elemene, emitefur, epirubicin, epristeride, estramustine analogue, estrogen agonists, estrogen antagonists, ctanidazole, ctoposide phosphate, exemestane, fadrozole, fazarabine, fenretinide, filgrastim, finasteride, flavopiridol, flezelastine, fluasterone, fludarabine, fluorodaunorunicin hydrochloride, forfenimex, formestane, fostriccin, fotemustine, gadolinium texaphyrin, gallium nitrate, galocitabine, ganirelix, gelatinase inhibitors, gemcitabine, glutathione inhibitors, hepsulfam, heregulin, hexamethylene bisacetamide, hypericin, ibandronic acid, idarubicin, idoxifenc, idramantone, ilmofosine, ilomastat, imatinib (Gleevec®), imiquimod, immunostimulant peptides, insulin-like growth factor-1 receptor inhibitor, interferon agonists, interferons, interleukins, iobenguane, iododoxorubicin, ipomeanol, 4-, iroplact, irsogladine, isobengazole, isohomohalicondrin B, itasetron, jasplakinolide, kahalalide F, lamellarin-N triacetate, lanreotide, leinamycin, lenograstim, lentinan sulfate, leptolstatin, letrozole, leukemia inhibiting factor, leukocyte alpha interferon, leuprolide+estrogen+progesterone, leuprorelin, levamisole, liarozole, linear polyamine analogue, lipophilic disaccharide peptide, lipophilic platinum compounds, lissoclinamide 7, lobaplatin, lombricine, lometrexol, lonidamine, losoxantrone, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, menogaril, merbarone, meterelin, methioninase, metoclopramide, MIF inhibitor, mifepristone, miltefosine, mirimostim, mitoguazone, mitolactol, mitomycin analogues, mitonafide, mitotoxin fibroblast growth factor-saporin, mitoxantrone, mofarotene, molgramostim, Erbitux, human chorionic gonadotrophin, monophosphoryl lipid A+myobacterium cell wall sk, mopidamol, mustard anticancer agent, mycaperoxide B, mycobacterial cell wall extract, myriaporone. N-acetyldinaline, N-substituted benzamides, nafarelin, nagrestip, naloxone/pentazocine, napavin, naphterpin, nartograstim, nedaplatin, nemorubicin, neridronic acid, nilutamide, nisamycin, nitric oxide modulators, nitroxide antioxidant, nitrullyn, oblimersen, O6-benzylguanine, octreotide, okicenone, oligonucleotides, onapristone, ondansetron, ondansetron, oracin, oral cytokine inducer, ormaplatin, osaterone, oxaliplatin, oxaunomycin, paclitaxel, paclitaxel analogues, paclitaxel derivatives, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, perillyl alcohol, phenazinomycin, phenylacetate, phosphatase inhibitors, picibanil, pilocarpine hydrochloride, pirarubicin, piritrexim, placetin A, placetin B, plasminogen activator inhibitor, platinum complex, platinum compounds, platinum-triamine complex, porfimer sodium, porfiromycin, prednisone, propyl bis-acridone, prostaglandin J2, proteasome inhibitors, protein A-based immune modulator, protein kinase C inhibitor, protein kinase C inhibitors, microalgal, protein tyrosine phosphatase inhibitors, purine nucleoside phosphorylase inhibitors, purpurins, pyrazoloacridine, pyridoxylated hemoglobin polyoxyethylene conjugate, raf antagonists, raltitrexed, ramosetron, ras farnesyl protein transferase inhibitors, ras inhibitors, ras-GAP inhibitor, retelliptine demethylated, rhenium Re 186 etidronate, rhizoxin, ribozymes, RII retinamide, rohitukine, romurtide, roquinimex, rubiginone B1, ruboxyl, safingol, saintopin, SarCNU, sarcophytol A, sargramostim, Sdi 1 mimetics, semustine, senescence derived inhibitor 1, sense oligonucleotides, signal transduction inhibitors, sizofiran, sobuzoxane, sodium borocaptate, sodium phenylacetate, solverol, somatomedin binding protein, sonermin, sparfosic acid, spicamycin D, spiromustinc, splenopentin, spongistatin 1, squalamine, stipiamide, stromelysin inhibitors, sulfinosine, superactive vasoactive intestinal peptide antagonist, suradista, suramin, swainsonine, tallimustine, tamoxifen methiodide, tauromustine, tazarotene, tecogalan sodium, tegafur, tellurapyrylium, telomerase inhibitors, temoporfin, teniposide, tetrachlorodecaoxide, tetrazomine, thaliblastine, thiocoraline, thrombopoietin, thrombopoietin mimetic, thymalfasin, thymopoietin receptor agonist, thymotrinan, thyroid stimulating hormone, tin ethyl etiopurpurin, tirapazamine, titanocene bichloride, topsentin, toremifene, translation inhibitors, tretinoin, triacetyluridine, triciribine, trimetrexate, triptorelin, tropisetron, turosteride, tyrosine kinase inhibitors, tyrphostins, UBC inhibitors, ubenimex, urogenital sinus-derived growth inhibitory factor, urokinase receptor antagonists, vapreotide, variolin B, velaresol, veramine, verdins, vinorelbine, vinxaltine, vitaxin, vorozole, zanoterone, zeniplatin, zilascorb, zinostatin stimalamer, or a combination thereof.

### Methods of Disrupting the YAP TAZ-TEAD Transcriptional Complex

YAP and TAZ kinase phosphorylation reactions are part of the broader Hippo signaling pathway that regulates cell proliferation apoptosis, and stem cell self-renewal. More particularly, Yes-associated protein (YAP)/transcriptional coactivator with a PDZ-binding domain (TAZ) are the prime mediators of the Hippo signaling pathway. The Hippo kinase cascade is the primary regulator of YAP/TAZ by phosphorylating YAP/TAZ and inhibiting their nuclear activities. YAP/TAZ, as a key regulator of the Hippo signaling pathway, serves as a nexus and integrator for multiple prominent pathways and signaling organelles that play key roles in the control of cell fate and tissue regeneration, such as Wnt, G protein-coupled receptor (GPCR), epidermal growth factor (EGF), bone morphogenetic protem (BMP)/transforming growth factor beta (TGFB), and Notch pathways.

There are two forms YAP/TAZ involved in the Hippo signaling pathway. One form is phosphorylated YAP/TAZ, which is phosphorylated directly by activated LATS½ kinases. In tum, multiple serine residues of YAP and TAZ are phosphorylated, leading to either cytoplasmic retention of YAP/TAZ via a 14-3-3 interaction or degradation by proteasomes. Another form of TAP/YAZ is bypophosphorylated (or unphosphorylated) YAP/TAZ When the kinase module of the Hippo signaling pathway is inactivated, bypophosphorylated YAP/TAZ translocates into the nucleus and induces related target gene expression. The entrance of hypophosphorylated YAP into the nucleus provides a transcriptional activation domain to its various binding partners, including the TEA domain family member TEAD proteins. While TEAD proteins (e g., TEAD1-TEAD4) can bind to DNA, they do not have a transcriptional activation domain, requiring Yap or WWtr/TAZ to regulate downstream target genes, many of which promote proliferation and survival.

Dysregulation of this pathway, in particular the YAP and TAZ downstream phosphorylation reactions, contributes to overgrowth of tissue/tissue fibrosis, tumor growth, cardiovascular disease, pulmonary hypertension, atherosclerosis, cardiac hypertrophy, musculoskeletal diseases (particular related to osteoclast formation), and the function of immune cells, among other things.

Verteporfin (VP) is a suppressor of YAP-TEAD complex and accordingly may be used to treat certain cancers and diseases. VP treatment can lead to the inhibition of proliferation of cancer cells and also to the suppression of migratory and invasive capacities of cancer cells. For example, a method of treating cancer may comprise administering to a subject the compositions described herein, wherein the cancer comprises a cell expressing YAP, TAZ, or a combination thereof, inclusive of wild-type and mutant cells. Further discussion of the Hippo pathway and the related use of photosensitizers is found, for example, in U.S. Pat. No. 11,331,304 and Feng et al., Verteporifin, a Suppressor of YAP-TEAD Complex, Presents Promising Antitumor Properties on Ovarian Cancer, ONCO. TARGETS THER. 2016; 9: 531-5381, both of which are herein incorporated in their entirety.

### Methods of Photodynamically Opening the Blood Brain Barrier for Drug Delivery

Photodynamic treatment (PDT) causes a significant increase in the permeability of the blood-brain barrier (BBB). Various combinations of laser radiation and photosensitizer may be used to reversibly open the BBB. The methods of opening the blood-brain barrier comprise, for example, (1) administering to a target location a composition comprising a plurality of amorphous nanoparticles comprising a photosensitizer in an amount effective to facilitate photodynamic therapy; and (2) exposing the amorphous nanoparticles to photoactivating light having a wavelength capable of being absorbed by the photosensitizer; wherein the exposure reversibly opens the blood-brain barrier, or a combination thereof.

In an embodiment, the radian may be provided at any suitable wavelength, for example between about 200 nm to about 1000 nm (e.g., 1-100 J/cm²). The photosensitizer may be provided in a concentration capable of effecting PDT, including, for example, between about 10 mg/kg and about 1000 mg/kg.

### Kits

The present disclosure encompasses kits to facilitate the administration of the compositions in a clinical setting. An example kit comprises a unit dosage form of the nanoparticles in any desirable form, and optionally an additional optional ingredient or other therapeutic agent. The kit may optionally further comprise a device for administering the nanoparticles, including a syringe, drip bag, patch, bottle, or inhaler.

Kits of the disclosure can further comprise a suitable vehicle that can be used to administer one or more active ingredients. For example, if the nanoparticles and/or additional optional ingredient and/or therapeutic agent are provided in a solid form that must be reconstituted for parenteral administration, the kit can comprise a sealed container of a suitable vehicle in which the active ingredient can be dissolved to form a particulate-free sterile solution that is suitable for parenteral administration. Examples of suitable vehicles include but are not limited to aqueous vehicles such as sodium chloride injection, dextrose injection, or a combination thereof, water-miscible vehicles such as ethyl alcohol, polyethylene glycol, and polypropylene glycol, and non-aqueous vehicles such as an oil (e.g., corn oil, cottonseed oil, peanut oil, sesame oil) ethyl oleate, isopropyl myristate, and benzyl benzoate.

### EXAMPLES

Embodiments of the present invention are further defined in the following non-limiting Examples. It should be understood that these Examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

Regarding the Examples described herein, results are presented in mean±standard error of the mean (SEM). Statistical tests were carried out using GraphPad Prism (GraphPad Software). Specific tests and the number of repeats are indicated in the figure captions. Reported P values are two-tailed. One-way ANOVA statistical tests and appropriate post hoc tests were carried out to avoid type-I errors.

### Example 1A. NanoVP Synthesis and Characterization

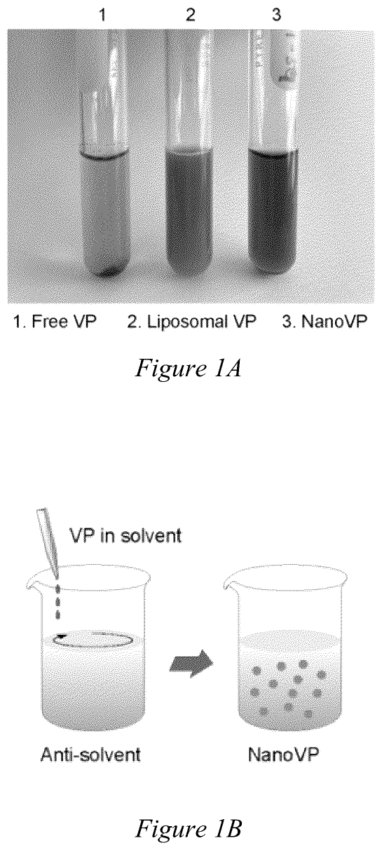

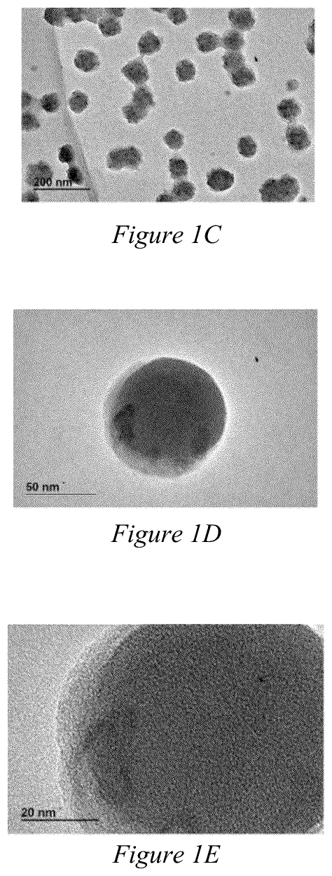

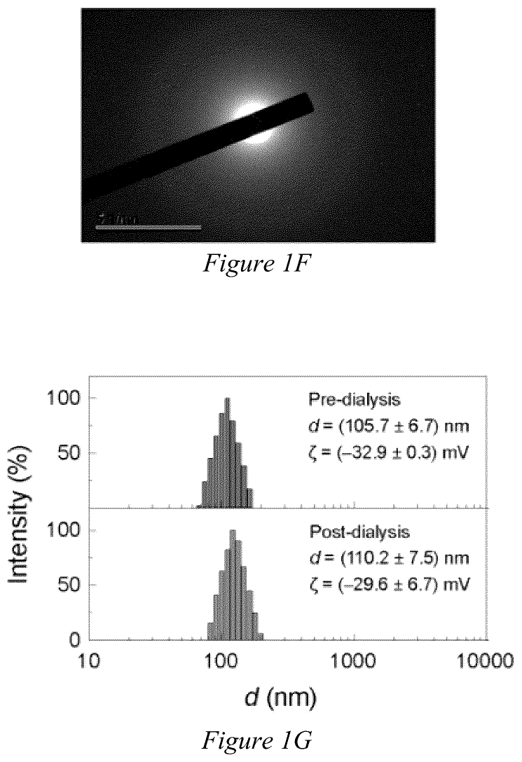

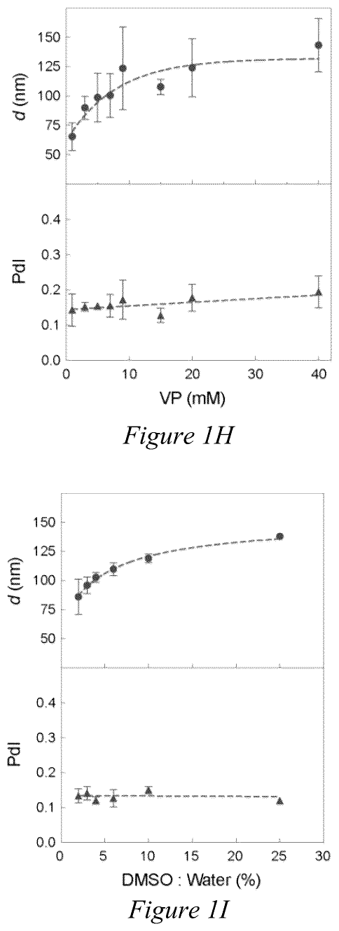

NanoVP was prepared using the solvent antisolvent precipitation method. Free-form verteporfin (VP) powders (US Pharmacopeia) were dissolved in dimethyl sulfoxide (DMSO, solvent) to achieve different initial concentrations (1-40 mM) and then injected dropwise into deionized water (antisolvent), under stirring (400 rpm), at room temperature. Different solvent- antisolvent ratios (1:50-1:10) were tested for formulating NanoVP. The results of this method are shown in FIG. 1A and FIG. 1B. As described herein, verteporfin is barely soluble in water and, as shown in FIG. 1A, is prone to forming J-type aggregates in physiologically relevant buffers. To date, intravenous delivery of verteporfin in the clinic relies on the use of liposome, which serves as a solubilizer. Beneficially, as shown in FIG. 1B a well-dispersed, carrier-free nano drug of verteporfin (NanoVP) can be prepared using the solvent-antisolvent precipitation technique. In particular, the dropwise addition of the sample results in amorphous solids. Without being bound by theory, it is thought that because DMSO and water are miscible, the introduction of VP DMSO solution in water creates local supersaturation of VP, which is thermodynamically unfavorable and potentially leads to the production of amorphous solids (precipitation) or crystals (crystallization).

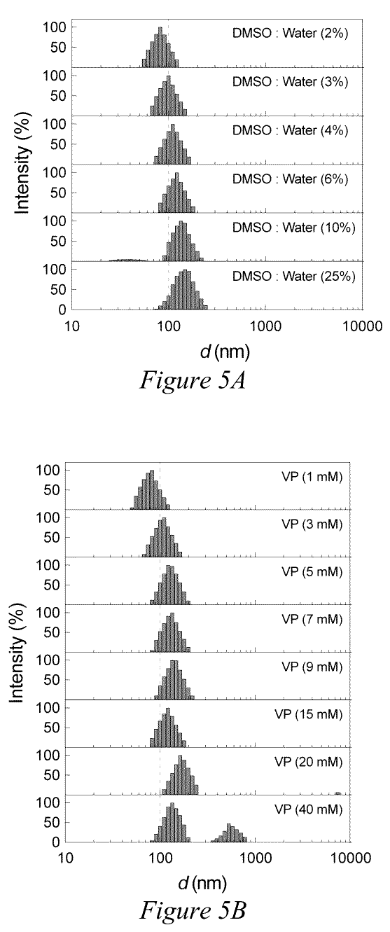

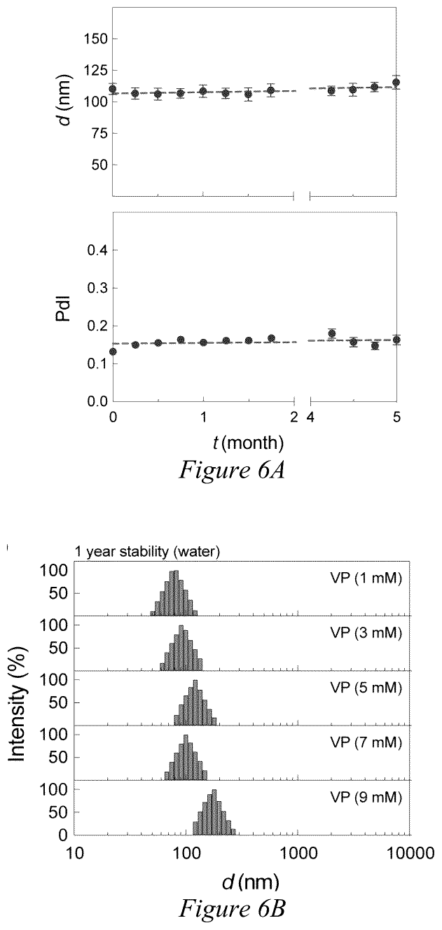

Free-form VP molecules not loaded into the NanoVP were then removed by dialysis (Spectrum Labs, MWCO 300 kDa) against phosphate-buffered saline (PBS) at 4° C. for 24 hours. The hydrodynamic diameter, polydispersity, and zeta potential were measured using a particle sizer and zeta potential analyzer (NanoBrook Omni, Brookhaven Instruments). VP concentration was determined based on UV-Vis absorbance in DMSO (Synergy Neo2, BioTek Instruments) using the established molar extinction coefficient (VP: ε=80,500 M-1 cm-1 at 435 nm, ε=34,895 M-1 cm-1 at 687 nm). Entrapment efficacy is the percentage of VP successfully encapsulated into the NanoVP. Loading capacity is the amount of VP loaded per unit weight of the NanoVP. NanoSight tracking analysis (NanoSight LM10, Malvern Instruments) was used to determine the number of particles per mL. Liposomes are considered ‘gold standard’ nanocarriers for VP delivery. NanoVP was synthesized using a 7 mM VP initial concentration and a 1:50 DMSO-to-water ratio was used for the remaining experiments. The results of the hydrodynamic diameter, polydispersity, and zeta potential testing are shown in FIG. 1G, FIG. 1H, FIG. 1I, FIGS. 5A-5B, and FIGS. 6A-6B.

As shown in FIG. 1G, dynamic light scattering and laser Doppler electrophoresis measurements showed that NanoVP has a hydrodynamic diameter of 105.7±6.7 nm (PdI: 0.13±0.1) and a zeta potential of −32.9±0.3 mV. Dialysis for purification and buffer exchange had minimal impact on NanoVP size (110.2±7.5 nm), PdI (0.13±0.2), and zeta potential (−29.6±6.7 mV). NanoVP has an entrapment efficacy of 91.6±7.5%, reflecting minimal loss during sample preparation and dialysis. The loading capacity of NanoVP is 100% at 706,000±38,000 VP molecules per nanoparticle, which is approximately 1455-fold greater than Liposomal VP (485±75 VP molecules per nanoparticle). Further, as reflected in FIG. 1H, the size of the NanoVP is tunable between 65 nm and 150 nm by increasing the photosensitizer concentration in solvent (1-40 mM) or, as shown in FIG. 1I, the DMSO:Water ratio (2-25%). However, as reflected in FIGS. 5A-5B, a starting VP concentration beyond 15 mM or a DMSO: Water ratio higher than 6% resulted in multi-peak size distribution. In comparison, as shown in FIGS. 6A-6B, NanoVP, synthesized using 1-7 mM VP and 2% DMSO:Water ratio, was found stable for over one year in water (pre-dialysis) and at least five months in PBS (post-dialysis).

### Example 1B. NanoVP Electrostatic Stabilization

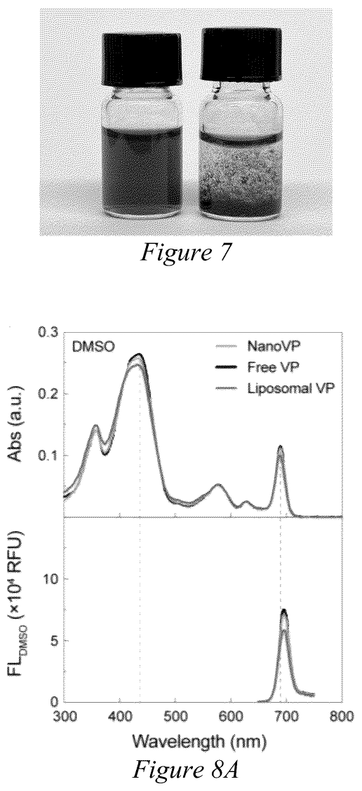

Further studies were conducted to assess NanoVP electrostatic stabilization. The electrostatic stability of NanoVP was probed using the neutral polymer, poloxamer 407 (Pluronic® F-127; Sigma), and the cationic polymer, polyethyleneimine (PEI). Polymers were dissolved in water according to the manufacturer's instructions and were added to the antisolvent (ultra-pure deionized water) prior to drop-wise synthesis or after NanoVP particles were formed, at a 1:5 polymer: VP ratio (w:w). As shown in FIG. 7, in both instances, the addition of positively charged PEI resulted in uncontrollable agglomeration, while NanoVP particles coated with poloxamer 407 appeared to remain stable. Dynamic light scattering (DLS) and laser Doppler electrophoresis (LDE) measurements showed that NanoVP coated with poloxamer 407 has a hydrodynamic diameter of 112.7±5.9 nm (PdI: 0.10±0.01) and zeta potential of −32.6±1.8 mV. DLS and LDE measurements cannot be accurately performed on large agglomerations. These results indicate that NanoVP stability is maintained via electrostatic repulsion forces between the particles.

### Example 1C. Comparison of NanoVP With Liposomes

Liposomes containing VP within phospholipid bilayers were synthesized via the freeze-thaw extrusion technique and used as a control group. Briefly, dipalmitoylphosphatidylcholine (DPPC), cholesterol, distearoylphosphatidylethanolamine-methoxy polyethylene glycol (DSPE-mPEG2000), and dioleoyltrimethylammoniumpropane (DOTAP; Avanti Polar Lipids) were mixed in chloroform at 6:3:0.3:0.7 molar ratio and co-dissolved with 50-200 nanomoles of VP (US Pharmacopeia). Chloroform was removed by a rotary evaporator to create a thin lipid film, which was rehydrated with 1 mL of ultrapure deionized water (Invitrogen). The phospholipid VP suspension was subjected to freeze-thaw cycles (4-45° C.). The dispersions were extruded through polycarbonate membranes (0.1 μm pore size; Avanti Polar Lipids) at 42° C. to form unilamellar vesicles. Liposomal VP samples were dialyzed against phosphate-buffered saline (PBS) at 4° C. and stored at 4° C. until use. The concentrations and loading capacity of Liposomal VP were determined by UV-Vis spectroscopy. Hydrodynamic diameter, polydispersity, and zeta potential of Liposomal VP were measured using a particle sizer and zeta potential analyzer (NanoBrook Omni, Brookhaven Instruments).

### Example 2. Transmission Electron Microscopy and Electron Diffraction

The size, morphology, and microstructure of NanoVP were studied using a transmission electron microscope (JEOL JEM-2100 LaB6, 200 kV). For conventional TEM, NanoVP (10 μL) was pipetted onto a Lacey carbon grid (Ted Pella) and air dried overnight before the examination. To exclude the possibility of artifacts due to sample dehydration, ionic liquid (3 μL, Hilem IL-1000, Hitachi) was used as a pretreatment reagent for TEM examination of wet specimens. Images were taken at high magnifications (10,000-100,000x). Due to small particle sizes and to enhance diffraction intensity, selected area electron diffraction was used to collect electron diffraction patterns.

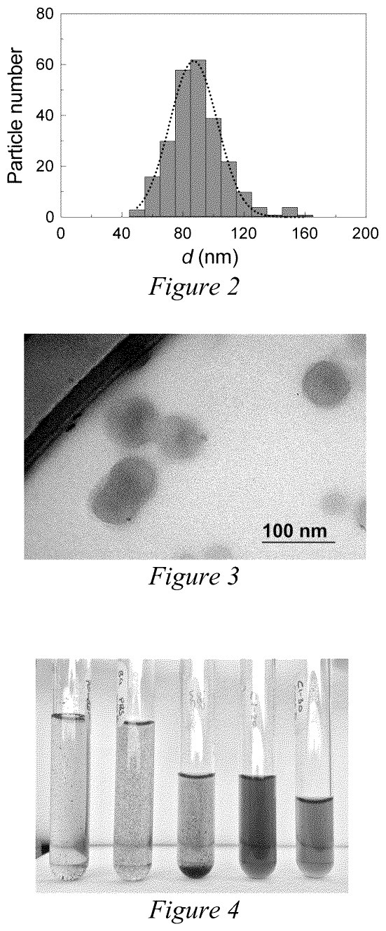

As shown in FIG. 1C-FIG. 1I and FIG. 2, TEM micrographs revealed that NanoVP particles are spherical-like, amorphous, monodispersed, and ˜100 nm in diameter. Further, as shown in FIG. 1F, the lack of electron diffraction further verified the amorphous structure of NanoVP. TEM study using ionic liquid treated samples and as depicted in FIG. 3, showed that sample dehydration, destined for conventional TEM, has a negligible influence on the structure and size of NanoVP. As shown in FIG. 4, other attempts at synthesis, including adding VP (in 100% DMSO) dropwise into PBS, resulted in uncontrollable agglomeration, potentially because the salts in PBS either served as nucleation sites or changed the charge of the dispersion.

### Example 3. NanoVP Stability, Photoactivity, and Photosensitizer Release Profile

The stability of NanoVP particles in PBS at 4° C. was determined by monitoring their hydrodynamic size and polydispersity index using a particle sizer analyzer for up to one year. NanoVP singlet oxygen (¹O₂) generation, self-quenching, and drug release were studied in 96-well plates as described previously. NanoVP and singlet oxygen sensor green (SOSG) at 5 μM were mixed and irradiated with 690 nm light (10 J/cm², 10 mW/cm²; ML6600, Modulight). A multi-mode microplate reader (Synergy Neo2, BioTek) acquired fluorescence signals of VP (Excitation/Emission: 435/650-750 nm) or SOSG (Excitation/Emission: 504/525 nm) before and after light irradiation. Self-quenching (FDSMO/FO) is defined as the fluorescence after disruption of the NanoVP using DMSO (FDSMO) divided by the fluorescence of the NanoVP in PBS (FO). Photosensitizer release from NanoVP (de-quenching) was studied by monitoring the gain in VP fluorescence signal in PBS/DMSO buffer (0-100% v/v) at room temperature or in Eagle's Minimum Essential Medium (EMEM, Cellgro) with 0-10% v/v fetal bovine serum (FBS, Gibco) at 37° C.

Fluorescence photoactivity was defined as the maximal fluorescence intensity (FL) of NC-VP and L-VP in PBS divided by the maximal fluorescence intensity (FL) of NC-VP and L-VP in DMSO. The absorbance spectrums of the constructs over the visible spectrum were determined in PBS and DMSO. The singlet oxygen production capabilities of the constructs were studied in 96-well plates as described previously. Photobleaching, defined as, the FL of the constructs in PBS post-light exposure, divided by the FL pre-light exposure in PBS, was carried out in a similar manner as the previous experiments. Drug dissolution was studied by suspending NC in a cell culture medium with and without 10% FBS at 37° C. The max FL measured at varying time points was divided by the max FL of the nanocrystals in DMSO. A summary of the photoactivity and photosensitizer release profile identified by this example follows.

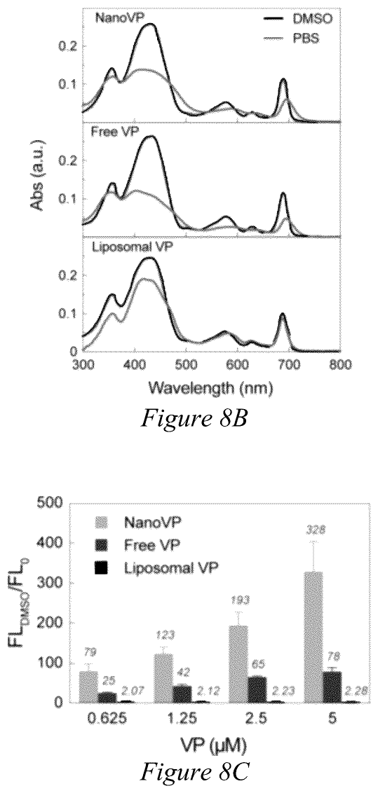

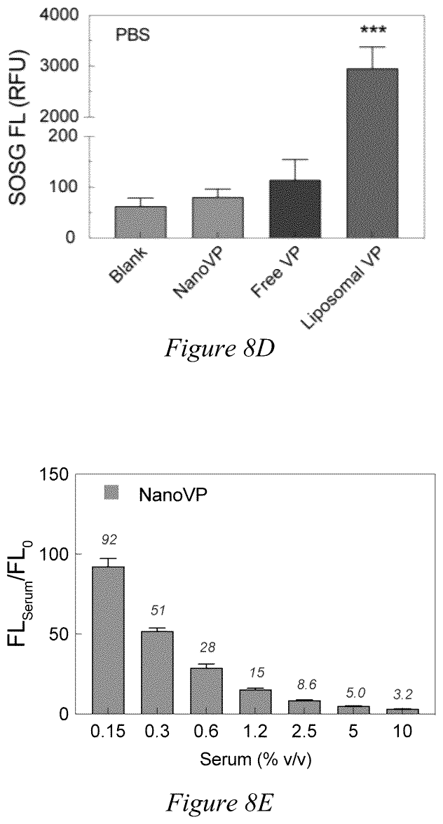

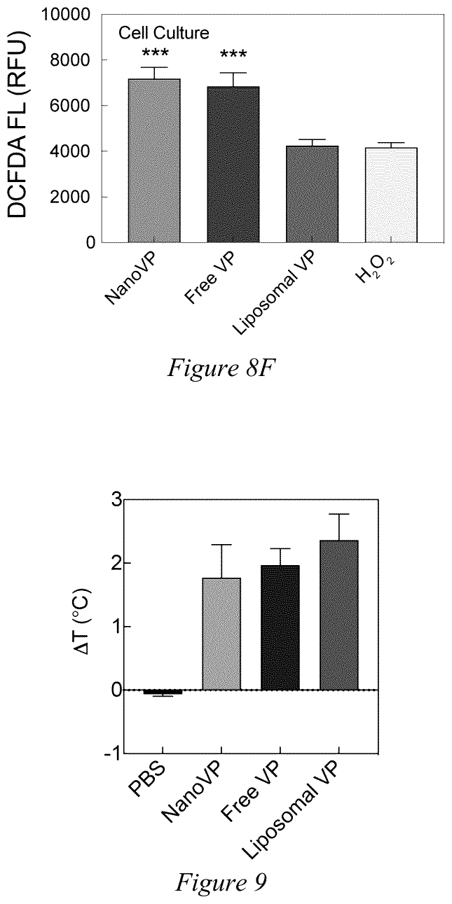

Verteporfin is a modified porphyrin derivative that displays a chlorin-type spectrum in organic solvents. As shown in FIG. 8A, in DMSO, the absorption spectrum of VP is characterized by a distinct Q band in the near-infrared region at 687 nm and a strong Soret band at 435 nm. The absorption spectra of NanoVP, freeform VP, and Liposomal VP were identical in DMSO, where VP is mainly in its monomeric form. At an excitation wavelength of 435 nm, the fluorescence emission spectra of NanoVP in DMSO can be recorded around 700 nm (see FIG. 8A). As shown in FIG. 8B, when NanoVP is well-dispersed in PBS, a broadening of the Soret band and a red-shifted Q band were observed. This suggests that monodispersed NanoVP consists of self-assembled J-aggregates. On the contrary, liposomes maintain some monomeric form of VP in PBS, showing a minimal decrease in the Soret band and without a red-shift displacement of the Q band as expected (see FIG. 8B). As shown in FIG. 8C, driven by the spatial confinement of high-number VP molecules in a 100 nm diameter particle, NanoVP generates extensive fluorescence self-quenching up to 328-fold in PBS in a concentration-dependent manner, where quenching=fluorescence in DMSO divided by fluorescence in PBS. The degree of self-quenching in free-form VP or Liposomal VP was only up to 78-fold or 2-fold, respectively. Verteporfin quenching can decrease the photochemical production of singlet oxygen (¹O₂) (a Type II reaction). As shown in FIG. 8D, upon light activation (690 nm, 10 J/cm², 10 mW/cm²), highly quenched NanoVP and free-form VP in PBS did not produce any significant amount of ¹O₂, as indicated by the minimal SOSG fluorescence signal. On the contrary, light activation of Liposomal VP generates an up to 34-fold higher SOSG signal, compared to NanoVP. Similar to that observed in clinical PDT practice and as depicted in FIG. 9, it was also confirmed that light activation of NanoVP, free-form VP, or Liposomal VP results in limited photothermal effects (ΔT=2-3° C.).

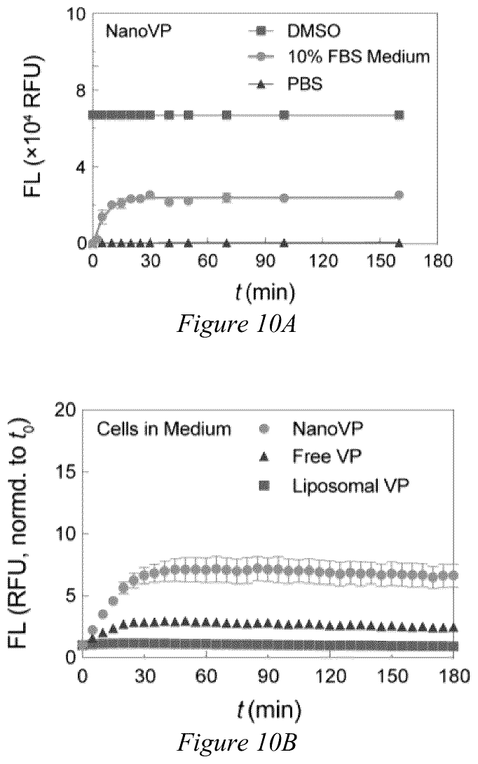

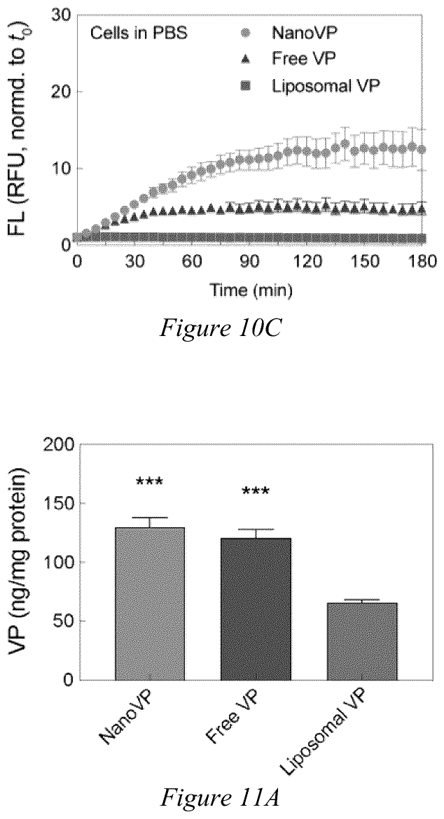

The highly quenched NanoVP can be unquenched in the presence of serum proteins and cancer cells for photosensitized ¹O₂ production. FIG. 8E shows that increasing the serum protein level from 0.15 to 10% v/v decreases NanoVP quenching from 92-fold to 3.2-fold. Disaggregation of NanoVP in serum containing media and U87 cells was further studied by longitudinal monitoring of the VP fluorescence signal; the results are shown in FIGS. 10A-10C. More specifically, as shown in FIG. 10A, after adding NanoVP to 10% v/v serum-containing media, a rapid increase in VP fluorescence within 30 minutes is followed by the plateau of the signal, whereupon the signal remains constant at 38% of that fully dissolved in DMSO. As reflected in FIG. 10B and FIG. 10C, similar trends were observed after adding NanoVP to U87 cells in serum-containing media or PBS, where fluorescence recovery was the highest in NanoVP samples, compared to free-form VP and Liposomal VP.

To check the recovery of photochemical activity of NanoVP in U87 cancer cells, DCFDA was used as a fluorescent probe for the detection of ROS generation. Intracellular reactive oxygen species (ROS) production was examined via 2′,7′-dichlorofluorescein diacetate (DCFDA; Thermo Fisher) assay. U87 cells were plated in 96-well black wall plates (Krystal) at a density of 2.2×104 cells/cm². On the next day, cells were incubated with a complete culture medium containing 0.25 μM of NanoVP, free-form VP, or Liposomal VP for 24 hours. On day 3, prior to photodynamic therapy (PDT), cells were washed twice with PBS and incubated with 10 μM DCFDA for 30 minutes. Cells were irradiated with 690-nm red light (0-10 J/cm², 50 mW/cm²; ML6600, Modulight). For the positive control, cells were incubated with 0.1 mL of 100 μM H₂O₂ for 15 minutes. The fluorescence signal of the cleaved DCF probe (Excitation/Emission: 485/535±15 nm) was measured using a microplate reader (Synergy Neo2, BioTek). The results are shown in FIG. 8F. Upon light activation (690 nm, 10 J/cm², 50 mW/cm²), disaggregated NanoVP produced significantly higher intracellular ROS (˜2-fold, P<0.001), compared to that of Liposomal VP and the positive control (100 μM H₂O₂).

### Example 4. Cell Cultures

Human glioblastoma U87 cell line and mouse 3T3 fibroblast cells were obtained from ATCC and cultured per the vendor's instructions. The human breast cancer MCF-7 parental cell line, the P-gp-overexpressing MCF-7 TX400 subline, and the ABCG2-overexpressing MCF-7 MX100 subline were cultured in EMEM supplemented with 10% FBS, 100 U/mL penicillin, 100 μg/mL streptomycin, and 0.01 mg/ml insulin (Sigma) as previously described. Cells were maintained in 5% CO₂ at 37° C. and tested to be free of mycoplasma (MycoAlert, Lonza).

### Example 5. Evaluation of Photosensitizer Uptake and PDT Responses in Vitro

Cells were cultured overnight in a 35-mm Petri dish or 96-well black wall plates (1-3.3×104 cells/cm²) and then incubated with photosensitizers (i.e., NanoVP, free-form VP, or Liposomal VP; 0.25 μM) for 24 hours. Subsequently, cells were washed twice with PBS and incubated with a photosensitizer free complete medium. Photosensitizer uptake in cells was determined using extraction methods followed by VP fluorescence measurements (Excitation/Emission: 435/700±10 nm, Synergy Neo2, BioTek) or visualized using fluorescence imaging (Lionheart, BioTek) as described previously. PDT was performed by exposing the cells to 690 nm light (0-10 J/cm², 10-50 W/cm², bottom illumination; ML6600, Modulight).

The generation of intracellular ROS was studied using 2′,7′-dichlorofluorescein diacetate probe (DCFDA, Thermo Fisher), and the mitochondrial membrane potential was examined via TMRE assay (tetramethylrhodamine ethyl ester, Abcam). More particularly, mitochondrial membrane potential (ΔΨm) was examined via tetramethylrhodamine ethyl ester (TMRE; Abcam) assay. U87 cells were plated in 96-well black wall plates at a density of 2.2×104 cells/cm². After a 24-hour incubation with 0.25 μM of NanoVP, free-form VP, or Liposomal VP, cells were washed twice with PBS and exposed to 690-nm light (0-10 J/cm², 50 mW/cm²). After PDT, cells were incubated with 250 nM TMRE for 25 minutes and washed twice with 0.2% bovine serum albumin (BSA) within PBS. Samples were subjected to fluorescence readings (Excitation/Emission: 549/575±10 nm) using a microplate reader or fluorescence and phase contrast imaging (Lionheart, BioTek). Controls include no light irradiation and incubation with 100 μM of carbonilcyanide p-triflouromethoxyphenylhydrazone (FCCP), a mitochondrial uncoupler, for 25 minutes. PDT and FCCP treated samples were normalized to a no-treatment control.

Expressions of total and cleaved caspase 3 were examined by immunoblotting techniques. U87 cells were plated in a 35-mm Petri dish (Falcon) at a density of 2.2×10⁴ cells/cm². Cells were incubated with 0.25 μM of NanoVP, free-form VP, or Liposomal VP for 24 hours. Cells were washed twice with PBS and irradiated with 690-nm light (10 J/cm², 50 mW/cm²) in a fresh culture medium. At 1 hour after PDT, cell lysates were collected in radioimmunoprecipitation assay (RIPA) buffer supplemented with a 1% protease inhibitor. Protein lysates (22 μg) were separated on a 4-12% precast Bis-Tris gel (NuPAGE) and transferred onto a nitrocellulose membrane. After blocking with Odyssey blocking buffer (Li-COR) for 1 hour, blots were incubated with primary antibody against beta-actin (#3700, cell signaling), caspase 3 (#9662, Cell Signaling,), or cleaved caspase 3 (#9661, Cell Signaling) overnight at 4° C. Blots were washed with 1X Tris-buffered Saline, 0.1% Tween® 20 Detergent (TBST) buffer followed by incubation with IRDye 680RD goat anti-mouse secondary antibody (926-68070, Li-COR) or IRDye 800CW goat anti-rabbit secondary antibody (926-32211, Li-COR) for 1 hour at room temperature based on the supplier's advice. Visualization of protein bands was developed using the LiCor ODYSSEY CLx (Li-COR). β-actin serviced as a loading control. Band intensity was determined using LiCOR ODYSSEY CLx software. PDT-treated samples were normalized to a no-treatment control.

At 24 hours after PDT, cell viability was determined using MTT [3-(4,5-dimethylthiazol-2-yl)-2,5-diphenyltetrazolium bromide] assay or neutral red assay (Abcam) following the vendor's protocol. For dark toxicity evaluation, cells were incubated with media containing photosensitizers (0-40 μM) for 72 hours, followed by the MTT assay. The photosensitizer efflux by ATP binding cassette (ABC) transporters was studied in MCF-7 and its multi-drug resistant sublines (MCF-7 TX400 and MCF-7 MX100) by adapting the protocols described herein.

In particular, to study ATP-binding cassette (ABC) transporter-mediated photosensitizer efflux, MCF-7, MCF-7 TX400, or MCF-7 MX100 were plated in 35 mm Petri dishes (Falcon) at a density of 3.3×10⁴ cells/cm². The next day, cells were incubated with 1 mM of NanoVP, free-form VP, or Liposomal VP for 4 hours at 37° C. with or without ABC drug transporter inhibitors (ABCG2 inhibitor: 10 mM fumetrimorgin C; P-gp inhibitor: 2.5 mM tariquidar). Subsequently, cells were washed twice with PBS, trypsinized, and lysed in RIPA buffer at 4° C. VP fluorescence (Excitation/Emission: 435/650-750 nm) was measured using a multimode microplate reader, and intracellular VP concentration was determined with appropriate VP standard curves. Intracellular VP was normalized to total cell protein. The total protein amount was determined using the Pierce BCA protein assay (Thermo Fisher).

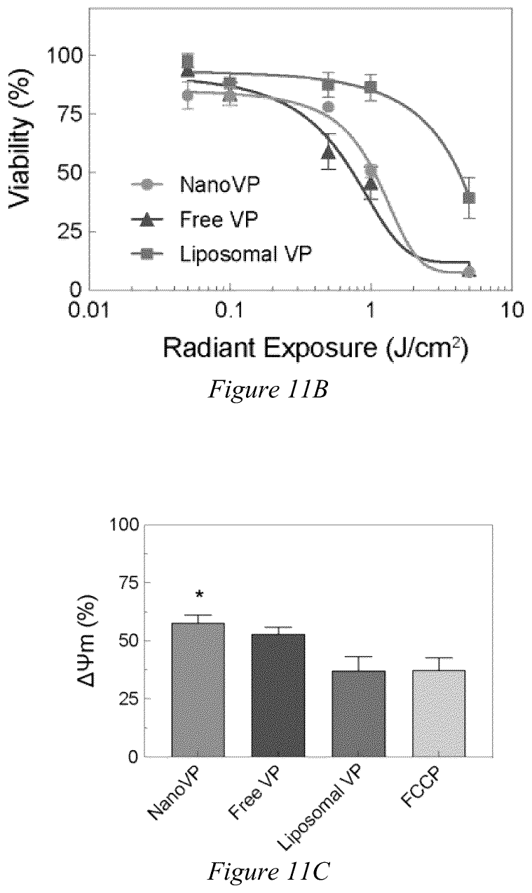

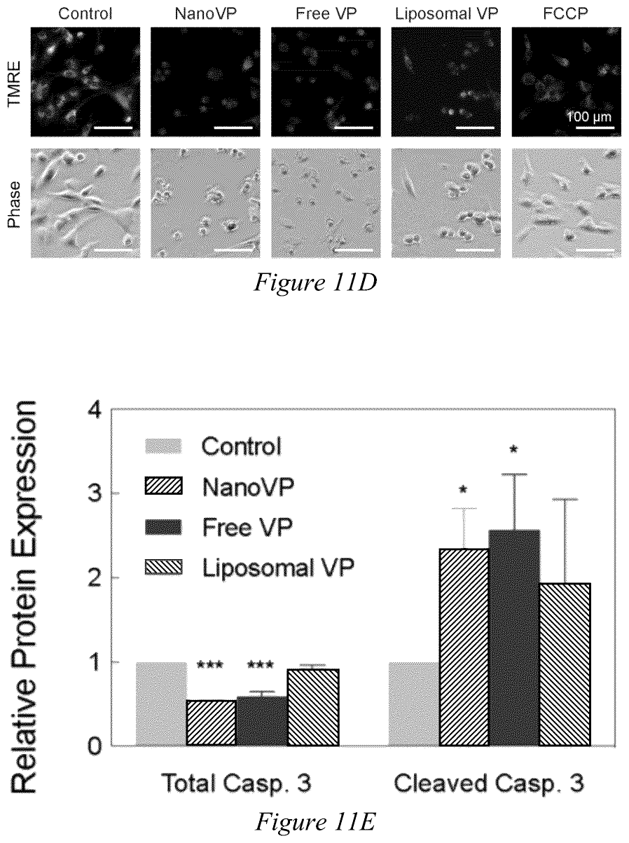

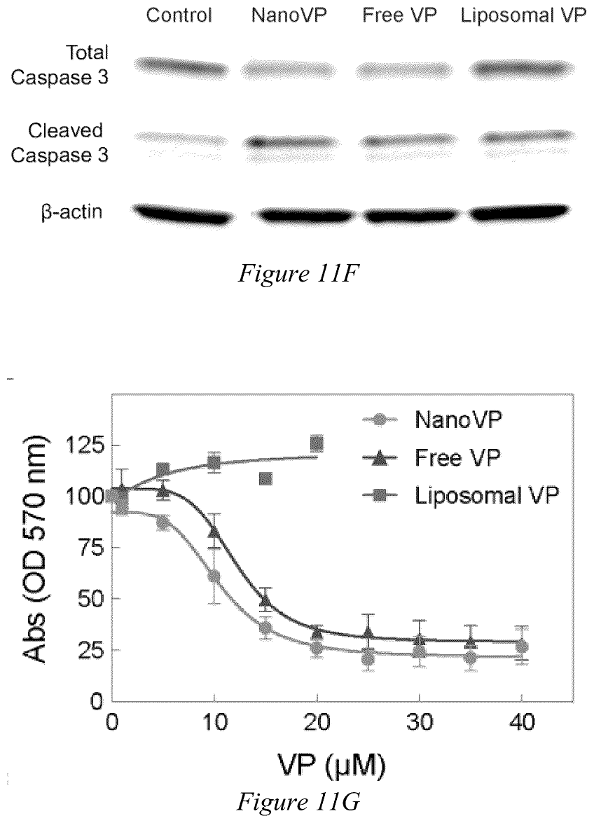

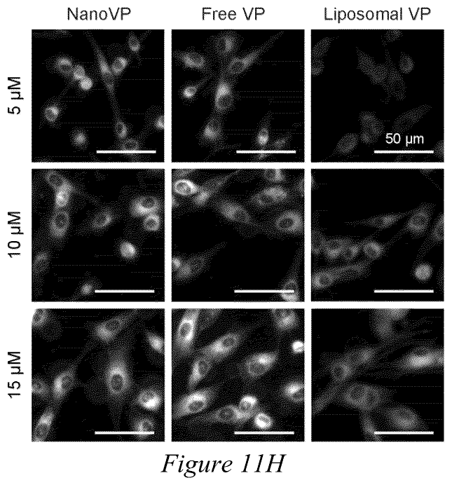

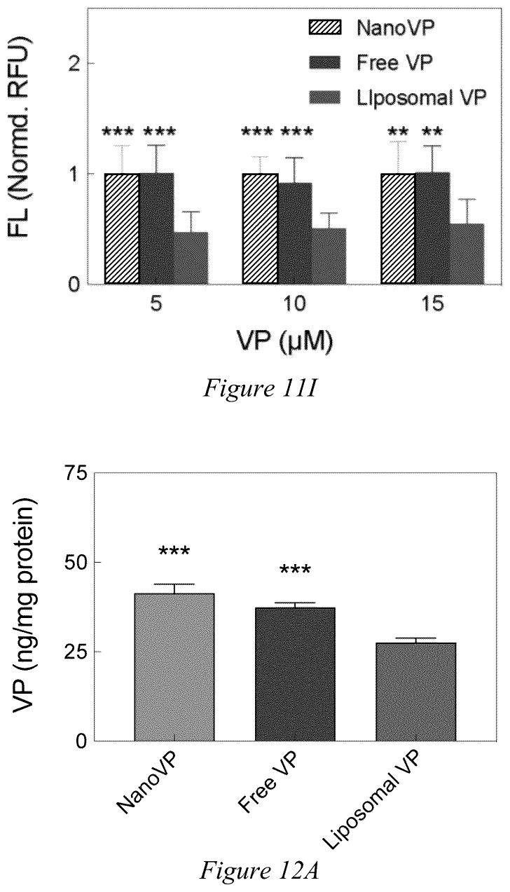

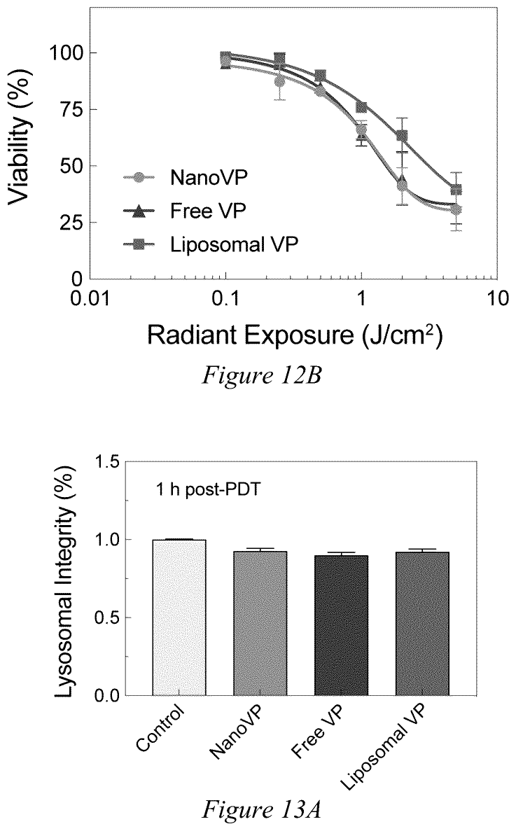

The therapeutic efficacy of PDT depends on the concentration of photosensitizer in cancer cells. As shown in FIG. 11A, U87 cells treated with NanoVP resulted in ˜2-fold higher intracellular concentration of VP (129.8±29.1 ng per mg of protein) compared to using Liposomal VP (66.2±8.1 ng per mg of protein). The uptake of NanoVP in U87 cells was similar to free-form VP. MTT assay, shown in FIG. 11B, revealed that NanoVP has a stronger anticancer PDT effect than Liposomal VP. NanoVP and free-form VP exhibited the lowest half maximal inhibition concentration (IC50) of 0.20 μM×J/cm² in U87 cells. At the same time, Liposomal VP displayed a 4-fold higher IC50 of 0.81 μM×J/cm². As reflected in FIGS. 12A-12B, the cellular uptake and PDT efficacy were further examined in non-cancerous 3T3 mouse fibroblast cells. The intracellular uptake of NanoVP in 3T3 cells was 49.9% greater than Liposomal VP. This resulted in a 2-fold IC50 increase from ˜0.4 μM×J/cm² for NanoVP to 0.8 μM×J/cm² for Liposomal VP. NanoVP-PDT in U87 cells has a 50% lower IC50 than 3T3 cells, while the IC50 of Liposomal VP-PDT remained similar in both U87 cancer 3T3 cells. These results indicate that the intracellular uptake and anti-cancer PDT efficacy of NanoVP is significantly higher than that of Liposomal VP.

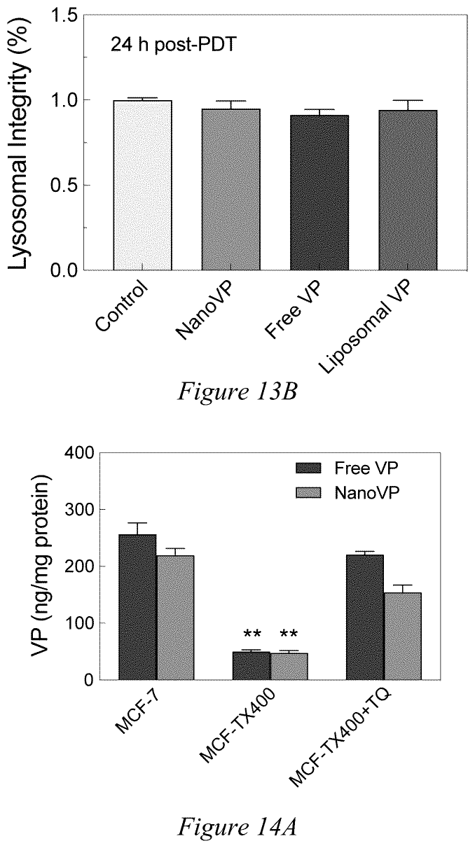

It has been shown that VP preferentially accumulates in mitochondria and light activation of VP depolarizes the mitochondrial membrane potential (ΔΨm) to trigger apoptosis. Light activation (690 nm, 10 J/cm², 50 mW/cm²) of NanoVP results in the largest degree of ΔΨm depolarization, as indicated by the tetramethylrhodamine ethyl ester (TMRE) assay, the results of which are shown in FIGS. 11C-11D. Quantification and normalization of the TMRE signal reveal NanoVP-PDT depolarizes ΔΨm by nearly 60%, while Liposomal VP-mediated PDT results in a modest depolarization of ΔΨm by ˜35%. The potent mitochondrial oxidative phosphorylation uncoupler, p-triflouromethoxyphenylhydrazone (FCCP), was used as a positive control. PDT-induced mitochondria damage can usually trigger the intrinsic apoptosis pathway leading to caspase-3 activation within a few hours after light activation. Immunoblotting results shown in FIGS. 11E-11F indicate that PDT using NanoVP or free-form VP induced a ˜2.4-fold increase in cleaved caspase-3 formation one-hour post light activation. In addition, NanoVP and free-form VP-mediated PDT significantly reduced total (pro) caspase-3 expression (i.e., non-activated caspase-3) by ˜50%, resulting in a cleaved-to-total caspase-3 ratio of ˜4.4. Liposomal VP-PDT resulted in the lowest ratio of ˜2.2. As reflected in FIGS. 13A-13B, the neutral red uptake (NRU) assay revealed that light-activation (690 nm, 1 J/cm², 50 mW/cm²) of NanoVP, free-form VP, or Liposomal VP does not affect lysosomal integrity in U87 cancer cells.

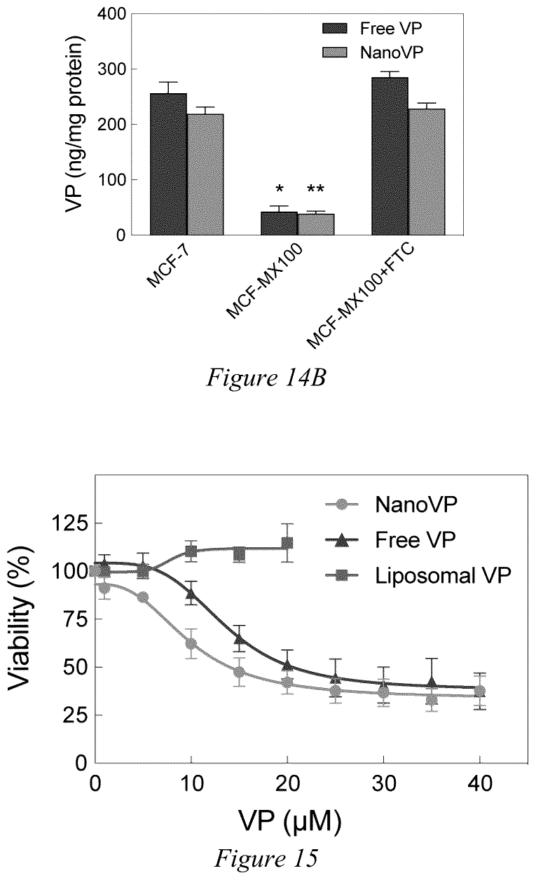

It has been shown that free-form VP can be effluxed by ATP-binding cassette (ABC) drug transporters (breast cancer resistance protein, ABCG2; P-glycoprotein, P-gp) in cancer cells and thereby reducing PDT efficacy. Using a human breast cancer cell line MCF-7, and their sub-lines (ABCG2 MCF-7 MX100 and or P-gp-overexpressing MCF-7 TX400), it was discovered that NanoVP is a substrate of ABCG2 and P-gp. These results are shown in FIGS. 14A-14B. The examples further demonstrated the use of ABCG2 inhibitor (fumitremorgin C, FTC) and P-gp inhibitor (tariquidar), can improve the accumulation of NanoVP in MCF-7 MX100 and MCF-7 TX400 cells, respectively (see FIGS. 14A-14B). In general, PDT requires very low doses of photosensitizers (nM−μM) to be effective. Recent clinical and preclinical studies have reported that high doses of VP (mM) can induce potent ‘dark’ toxicity (without light activation) in cancer cells, including GBM (NCT04590664). In complete darkness, NanoVP exhibited an IC50 of 12-14 μM in U87 cells and 3T3 cells, similar to free-form VP (see FIG. 11G and FIG. 15). NanoVP treatment at, or above, 20 μM significantly reduced cell viability by 75%. In contrast, as shown in FIGS. 11H-11I, liposomal VP exhibited no significant cytotoxicity, presumably due to the poor intracellular VP uptake.

### Example 6. In vivo PDT and Photosensitizer Biodistribution

Animal protocols were approved by the University of Maryland, College Park Institutional Animal Care, and Use Committee. Xenograft mouse models of glioblastoma were established by subcutaneously injecting U87 cells (1×106 cells in PBS/MatrigelR) into the flank of J:NU mouse (4-5 weeks old, #007850, Jackson Laboratory). Tumor volumes were longitudinally monitored using calipers and calculated using the standard estimation formula, V=½×length×width², where length equals the maximum tumor diameter in millimeters and width equals the diameter that is perpendicular to the length. Treatments were initiated 2 weeks post-implantation when tumors reached ˜100 mm3. At 2 hours post-intravenous injection of photosensitizers (i.e., NanoVP or Liposomal VP; 0.25 mg/kg) or PBS, a vertical 690 nm laser beam (100 J/cm², 100 mW/cm²; ML6600, Modulight) was focused on the tumor to activate PDT. A cloth was used to protect animal skin from light exposure.

Change in tumor volume was monitored for up to 2 months. The specific growth rate (SGR) of tumors was estimated using the equation (1/V)(dV/dt), where V is tumor volume and/is time. To examine photosensitizer biodistribution, tumor and normal tissues were collected at 2- and 24 hours post-injection of photosensitizers. Fluorescence images of VP in organs, tumors, and standards containing known VP concentrations were acquired using a reflectance fluorescent microscope equipped with a 375 nm laser diode (6 mW, L375P70MLD, Thorlabs), a 692 nm filter (FF01-692/40-25 Semrock), and a 12-bit CCD camera (EM-CCD, PCO, Kelheim, Lower Bavaria, Germany). Fluorescence images of VP in organs and tumors were processed using ImageJ software (NIH). 20 randomly selected points on the imaged organ were chosen and used to obtain the average intensity and standard deviation. After obtaining these values, the signal was converted to concentration through the use of the standard curves.

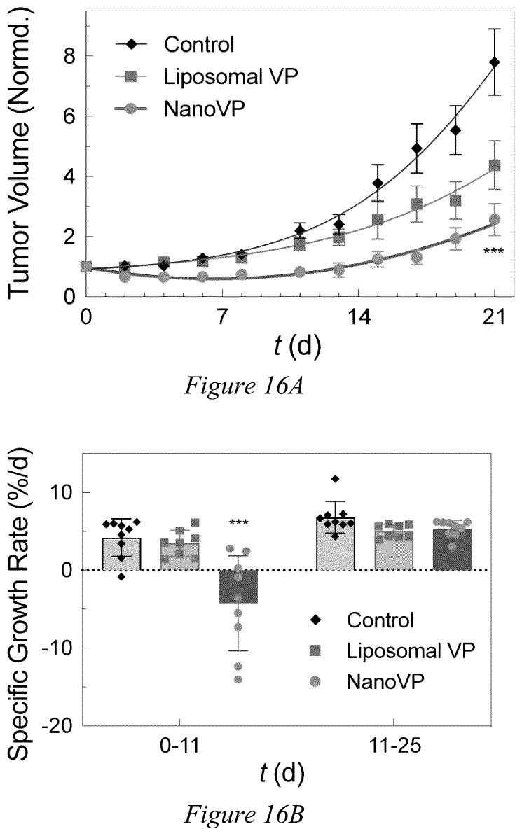

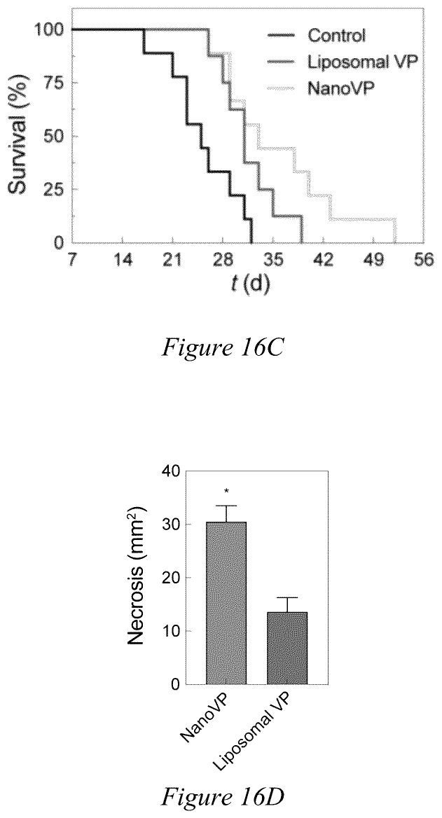

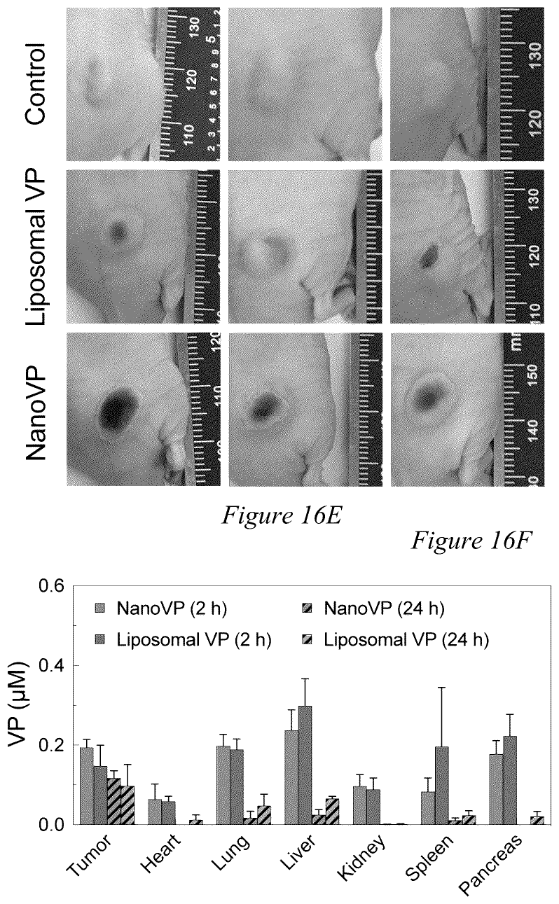

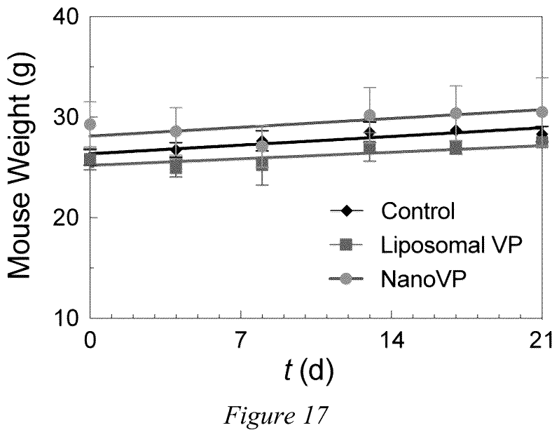

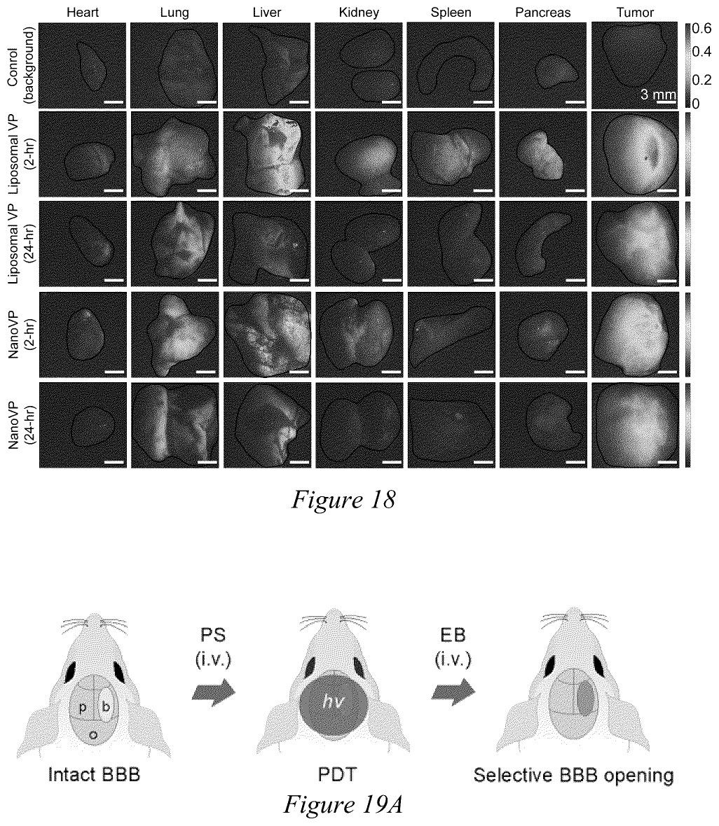

To assess the efficacy of NanoVP-PDT in controlling GBM tumors in vivo, PDT was performed fourteen days following implantation of U87 cells in mice, when tumors reached approximately 100 mm³ in volume. The results are shown in FIGS. 16A-16F. Liposomal VP-PDT served as the clinical gold standard. Light (690 nm) irradiation for PDT was performed two hours after a single intravenous injection of NanoVP or Liposomal VP at 0.5 mg/kg. At 2-11 days after treatment. NanoVP-PDT reduced tumor volume by up to 35%. In contrast, as shown in FIG. 16A, continued tumor growth was observed in ‘no-treatment’ control and ‘Liposomal VP-PDT’ over this same period. At 21 days post-treatment, the mean tumor volume reduction in mice treated with ‘NanoVP-PDT’ and ‘Liposomal VP-PDT’ was 70% and 44%, respectively, compared to ‘no-treatment’ control animals. These results suggest that NanoVP is a superior anti-tumor PDT agent compared to Liposomal VP. As shown in FIG. 16B, further analysis revealed that animals treated with ‘NanoVP-PDT’ experienced a significant tumor growth inhibition at a specific growth rate (SGR) of around −4.3%/day between days 0-11 after treatment. With only a single cycle of treatment, tumor regrowth can be observed 11 days after ‘NanoVP-PDT’ with an SGR of approximately 5.3%/day, similar to that of ‘no-treatment’ and ‘Liposomal VP-PDT’. As shown in FIG. 17, in ‘NanoVP-PDT’ and ‘Liposomal VP-PDT’ treated animals, the change in mouse weight was consistent with ‘no-treatment’ mice, indicating that PDT does not appreciably add to the systemic toxicity. It is noteworthy that a single cycle of treatment only modestly improved animal survival after ‘NanoVP-PDT’ (median survival: 33 days) and ‘Liposomal VP-PDT’ (median survival: 31 days), compared to ‘no-treatment’ control (median survival: 25) (See FIG. 16C). These results point out challenges in achieving meaningful improvements in treatment response for GBM and highlight the need for combination strategies developed to provide durable tumoricidal control in the future. In addition to changes in tumor volume, noticeable visual differences on tumor surfaces were observed (See FIG. 16D). At six days posttreatment, ‘NanoVP-PDT’ resulted in a significantly larger dark, possibly necrotic, area that covered the majority of the tumor (30.5 mm2), while ‘Liposomal VP-PDT’ led to a smaller (13.6 mm2) and more localized necrotic patch (See FIG. 16D, FIG. 16E). Finally, as shown in FIG. 16F and FIG. 18, fluorescence imaging of VP in mouse tissues revealed that NanoVP and Liposomal VP have a similar biodistribution profile at 2 and 24 hours post intravenous injection.

### Example 7. Photodynamic Opening of the Blood-Brain Barrier in Rodents

Sprague-Dawley rats (4-5 weeks old, Envigo) received an intravenous injection of NanoVP (0.25 or 0.5 mg/kg) or 5-aminolevulinic acid (5-ALA, 20 or 125 mg/kg) at 30 min or 4 hours before PDT. PDT parameters, including photosensitizer concentration, drug-light interval, irradiance, and radiant exposure, were selected. At 30 minutes post-photosensitizer injection, rats were anesthetized with isoflurane and secured within a stereotaxic frame.

During the 30-minute drug-light interval, a craniotomy was performed. The top of the rat head was shaved, and the skin was removed. The periosteum was retracted to the edges of the skull. Using a micro drill (OmniDrill35, WPI), a circle about 4 mm in diameter was sketched into the right parietal bone. Drilling was stopped periodically, and PBS was applied to prevent heating. The drilled bone was removed, and any bleeding that occurred from surgery was stopped prior to light irradiation. After a 30-minute circulation period with Evans blue, rats were sacrificed via isoflurane overdose and decapitation, and brains were harvested. Digital images were immediately captured, and the brains were frozen at −80° C.

PDT was performed by light activation of the exposed brain (NanoVP: 690 nm, 80 or 100 J/cm²; 5-ALA: 635 nm, 60 or 80 J/cm²; 40 or 85 mW/cm²; ML6600, Modulight). At 90 minutes post-PDT, rats received an intravenous injection of Evans blue dye (2%, 4 mL/kg) to determine the blood-brain barrier integrity using imaging and extraction methods In particular, Evans blue was quantified. The brain hemispheres were thawed on ice, minced, and incubated with formamide for 30 minutes at 55° C. followed by 4 days at room temperature. Samples were centrifuged to pellet the tissue, and the optical density of Evans blue within the supernatant at 610 nm was measured on a multi-mode microplate reader. Standard curves were used to determine the amount of Evans Blue in each brain hemisphere.

In a small group of PDT animals that did not receive Evans blue injection, brain tissues were collected, sectioned, and processed for (i) histological (hematoxylin and cosin stain, H&E) analysis and (ii) Luxol Fast Blue staining of myelin/myelinated axons and Nissl bodies. H&E and immunohistochemistry slides were imaged using the whole slide Aperio AT2 scanner system (Leica Biosystems).

All image analysis was accomplished using Aperio and Halo imaging analysis software (v3.3.2541.300; Indica Labs), and image annotations were performed by a pathologist (B. K). Fields were excluded if they contained large areas of artifact such as folds or tears. More particularly, The effects of PDT on healthy brain tissues were examined 2 hours after light irradiation. Brains were harvested and frozen within optimum cutting temperature (OCT) compound (Tissue-Tek) at −80° C. Cryocut sections (5 μm) from frozen rat brains were collected at the level of rostral and caudal to the treatment area and fixed in a mixture of glutaraldehyde and formaldehyde. Hematoxylin and cosin (H&E) staining was performed using the Sakura R Tissue-TekR Prisma automated stainer. The slides were hydrated and stained with commercial hematoxylin, clarifier, bluing reagent, and cosin-Y. A regressive staining method was used. This method intentionally overstains tissues and then uses a differentiation step (clarifier/bluing reagents) to remove excess stains. The slides were cover-slipped using the Sakura Tissue-Tek Prisma automatic cover slipper and dried prior to review. For Luxol Fast Blue staining, sections were hydrated and stained overnight at room temperature. Sections were immersed in 95% alcohol to remove excess stains. Slides were then washed in distilled water and quickly immersed in 0.05% lithium carbonate. Tissue sections were then placed in 70% ethanol until a sharp contrast between the corpus callosum and cortex developed. Sections were washed in distilled water, stained with cresyl violet acetate, differentiate in several changes of 95% alcohol, dehydrated with ethanol, cleared in xylene, and mounted. Slides were scanned at 20× using an Aperio AT2 scanner (Leica Biosystems) into whole slide digital images.

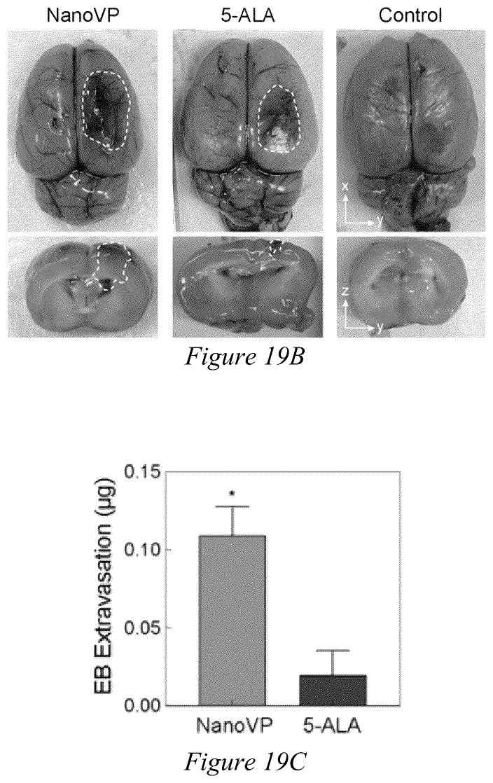

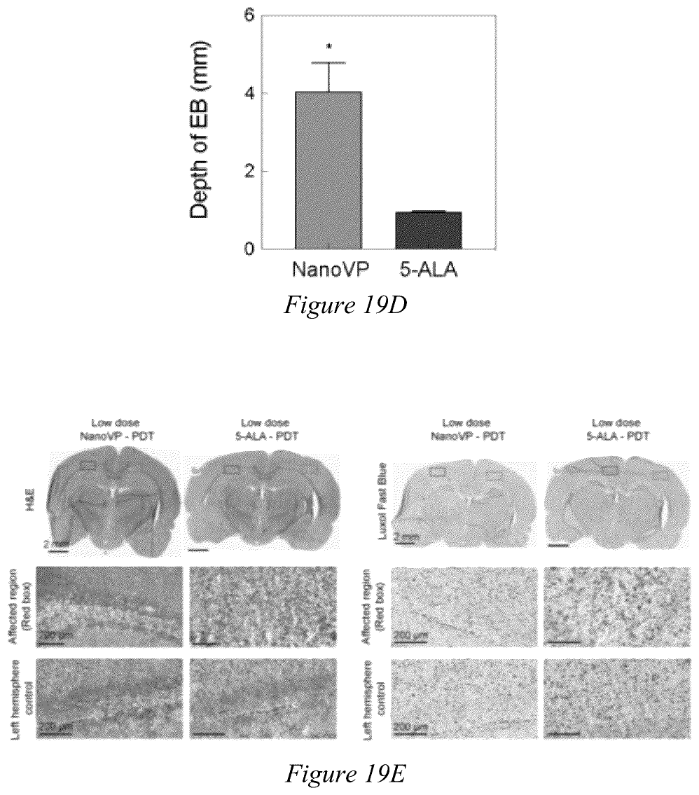

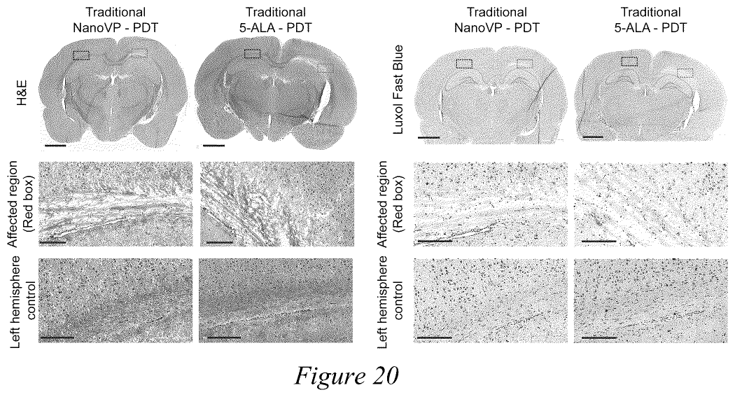

The blood-brain barrier (BBB) remains a critical obstacle to the effective treatment of GBM and other central nervous system diseases. Low-dose PDT priming, an enabling technology for minimally invasive tumor treatments, allows transient opening of the BBB through modulating endothelial cell-cell junction phenotype. The utility and safety of low-dose NanoVP-PDT priming for spatially targeted BBB opening in healthy rats with intact BBB were assessed, as shown in FIG. 19A. The 5-ALA (GliolanR), an FDA-approved photosensitizer prodrug routinely used for PDT opening of the BBB, served as a benchmark group. As shown in FIG. 19B and FIG. 19C, there was a 5.5-fold increase of total Evan Blue (a model drug) accumulation in NanoVP-PDT treated brain compared to 5-ALA-PDT treated brain tissues (P<0.05). The spatiotemporal selectivity of PDT confines Evan Blue delivery to the right-brain hemisphere where the light is directed, thereby reducing normal tissue damage. Further, as shown in FIG. 19D, the use of the longer activation wavelength, 690-nm, to photoactivate NanoVP for deeper tissue penetration is also an advantage over using 5-ALA-induced protoporphyrin IX, photoactivatable at 635-nm. It was demonstrated that 690-nm light activation of NanoVP improves Evan Blue delivery at further depths in rat brains (visible up to 4-5 mm), compared to 635 nm light activation of 5-ALA-induced PpIX (visible up to 1 mm). Histological changes of brain tissue after PDT were studied microscopically on sections stained with H&E or Luxol Fast blue. Compared to the left-brain hemisphere that did not receive treatment due to the intact skull, the right-brain hemisphere did not have any signs of low-dose PDT priming-induced damage. H&E staining revealed no detectable lesions at the site of treatment (right hemisphere) and, Luxol fast blue staining was symmetrical with no evidence of demyelination (see FIG. 19E). Alternatively, as shown in FIG. 20, rats that received traditional high-dose PDT had signs of brain damage within the neuropil parenchyma (brain cortex). This result agrees with previous studies that show dose escalation results in increased signs of edema and brain damage.

The embodiments being thus described, it will be obvious that the same may be varied in many ways. Such variations are not to be regarded as a departure from the spirit and scope of the disclosure and all such modifications are intended to be included within the scope of the following claims.

1-20. (canceled)

21. A nanoparticle composition comprising:
one or more amorphous nanoparticles comprising up to 100% by weight of a photosensitizer; wherein the one or more amorphous nanoparticles have a size of 1000 nm or less; and wherein the one or more amorphous nanoparticles are excipient-free, polymer-free, and lipid-free.

22. The nanoparticle composition of claim 21, wherein the photosensitizer absorbs light in a range of from about 400 nm to about 1200 nm.

23. The nanoparticle composition of claim 21, wherein the photosensitizer comprises a porphyrin photosensitizer.

24. A method of preparing a nanoparticle composition, said method comprising:
1) preparing a first liquid phase solution comprising a photosensitizer in a solvent;
2) preparing a second liquid phase solution comprising an antisolvent;
3) adding the first liquid phase solution dropwise into the second liquid phase solution; and
4) removing any remaining solvent, antisolvent, and photosensitizer to produce one or more excipient-free, amorphous nanoparticles.

25. The method of claim 24, wherein step (3) further comprises adding energy through mixing, homogenization, countercurrent flow homogenization, microfluidization, or a combination thereof.

26. The method of claim 24, wherein the photosensitizer is present in the first liquid phase solution in a concentration of between about 100 micromolar (μM) to about 100 millimolar (mM).

27. The method of claim 24, wherein the ratio between the solvent and the antisolvent is between about 1:50 to about 1:4.

28. The method of claim 24, wherein the antisolvent comprises water, an aqueous solution, or a combination thereof.

29. The method of claim 24, wherein the solvent comprises a water-miscible protic compound.

30. The method of claim 24, wherein the solvent is an aprotic organic solvent.

31. A method of treating a disease comprising:
administering to a target a therapeutically effective amount of a composition comprising the nanoparticle composition of claim 21.

32. The method of claim 31, wherein the target is a tumor, a tissue, a cell, a blood vessel, or a combination thereof.

33. The method of claim 32, wherein the tumor is a glioblastoma or a central nervous system tumor.

34. The method of claim 31, wherein the method of administration is intravenous, intratissue, intraperotoneal, topical, oral, mucosal, patenteral, transdermal, or a combination thereof.

35. The method of claim 31, further comprising the step of:
exposing the nanoparticle composition to photoactivating light having a wavelength capable of being absorbed by the photosensitizer;
wherein the exposure produces a cytotoxic reactive oxygen species at the target, opens the blood-brina barrier, or a combination thereof.

36. The method of claim 31, wherein the disease is a cancer.

37. The method of claim 36, wherein said cancer is brain cancer, neck cancer, skin cancer, eye cancer, oral cancer, head and neck cancer, blood cancer, bone cancer, or a combination thereof.

38. The method of claim 36, wherein said cancer comprises a cell expressing YAP, TAZ, or a combination thereof, inclusive of wild-type and mutant cells.
